(19) United States
(12) Patent Application Publication
(10) Pub. No.: US 2013/0045446 A1
(43) Pub. Date: Feb. 21, 2013

(54) RADIATION-SENSITIVE RESIN COMPOSITION, METHOD FOR FORMING A RESIST PATTERN AND SULFONIUM COMPOUND

(71) Applicants: Mitsuo SATO, Tokyo (JP); Kazuo NAKAHARA, Tokyo (JP)

(72) Inventors: Mitsuo SATO, Tokyo (JP); Kazuo NAKAHARA, Tokyo (JP)

(73) Assignee: JSR CORPORATION, TOKYO (JP)

(21) Appl. No.: 13/600,874

(22) Filed: Aug. 31, 2012

Related U.S. Application Data
(63) Continuation of application No. PCT/JP2011/054969, filed on Mar. 3, 2011.

(30) Foreign Application Priority Data
Mar. 3, 2010 (JP) 2010-047039

(51) Int. Cl. C07C 321/10; G03F 7/20; G03F 7/027

(52) U.S. Cl. 430/285.1; 430/281.1; 430/325; 560/227; 560/117

(57) ABSTRACT

A radiation-sensitive resin composition includes a sulfonium compound represented by a general formula (1), and a first polymer that serves as a base resin. R represents a group represented by a general formula (2). n₁ to n₃ each independently represent an integer of 0 to 5 and n₁+n₂+n₃≧1. X⁻ represents an anion. The sulfonium compound is preferably a compound represented by a following general formula (1-1).

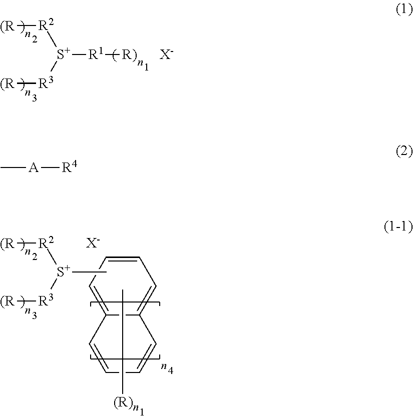

### CROSS-REFERENCE TO RELATED APPLICATIONS

The present application is a continuation application of International Application No. PCT/JP2011/054969, filed Mar. 3, 2011, which claims priority to Japanese Patent Application No. 2010-047039, filed Mar. 3, 2010. The contents of these applications are incorporated herein by reference in their entirety.

### BACKGROUND OF THE INVENTION

1. Field of the Invention

The present invention relates to a radiation-sensitive resin composition, a method for forming a resist pattern, and a sulfonium compound.

2. Discussion of the Background

In the microfabrication field in which integrated circuit elements are produced, a lithography technique which enables a micro fabrication at a level of no greater than 0.10 μm in order to obtain higher integrity has been earnestly desired. However, in conventional lithography techniques, a near ultraviolet ray such as an i-line as a radioactive ray is used. With a near ultraviolet ray, microfabrication at a level of no greater than 0.10 μm (sub quarter micron level) is extremely difficult to carry out. Accordingly, in order to facilitate microfabrication at a level of no greater than 0.10 μm, lithography technique using a radioactive ray having a shorter wavelength has been developed. Examples of the radioactive ray having a shorter wavelength include a far ultraviolet ray such as a bright line spectrum in a mercury lamp and an excimer laser, an X-ray, an electron beam, and the like. Among them, a KrF excimer laser (wavelength: 248 nm) and an ArF excimer laser (wavelength: 193 nm) have attracted attention.

With the attention on the excimer lasers, a number of materials of photoresist films for excimer lasers have been is proposed. Examples of such photoresist materials for excimer lasers include a composition (hereinafter, may be also referred to as “chemical amplification-type resist”) containing a component having an acid-dissociable functional group and a component that generates an acid (hereinafter, may be also referred to as “acid generating agent”) by irradiation of a radioactive ray (hereinafter, may be also referred to as “exposure”) and utilizing chemically amplified effects thereof, and the like. As a chemical amplification-type resist, specifically, a composition containing a resin having a t-butyl ester group of a carboxylic acid or a t-butyl carbonate group of phenol, and an acid generating agent has been reported. In the composition, a t-butyl ester group or t-butyl carbonate group existing in the resin dissociates by an action of an acid generated by the exposure, whereby the resin has an acidic group including a carboxyl group or a phenolic hydroxyl group. As a result, an exposed region of a photoresist film becomes readily soluble in an alkaline developer, which enables a desired resist pattern to be formed.

On the other hand, these days, in the microfabrication field, it is earnestly desired to form a further fine resist pattern (for example, fine resist pattern having a line width of about 45 nm). In order to allow a further fine resist pattern to be formed, for example, shortening of a light source wavelength in an lithography device as well as an increase of a numerical aperture (NA) of lens, and the like is would be conceived. However, shortening a light source wavelength requires a new lithography device, and such a device is expensive. In addition, there is a drawback that a depth of focus decreases even if a resolution can be improved, since a resolution has a trade-off relationship with a depth of focus in the case where the numerical aperture (NA) of lens is increased.

Accordingly, in recent years, as a lithography technique for solving the foregoing problems, a method referred as a liquid immersion lithography method has been reported. In the method, a liquid for immersion lithography (for example, pure water, fluorine-based inert liquid, or the like) is interposed between a lens and a photoresist film (on a photoresist film) upon exposure. According to the method, since a light path space for the exposure, which is conventionally filled with an inert gas such as air, nitrogen or the like, is filled with the liquid for immersion lithography having a higher refractive index (n) than the air and the like. Thus, a similar effect can be obtained to the case where a light source wavelength in the lithography device is shortened, i.e., a high resolving ability, even if a conventional exposure light source is used. In addition, there arises no problem of a decrease in a depth of focus.

Therefore, according to the liquid immersion lithography process, a resist pattern can be formed which is low in cost, excellent in resolving ability, and further excellent also in is a depth of focus using a lens mounted on an existing device. A number of compositions for use in such a liquid immersion lithography process have been disclosed (for example, see PCT International Publication No. 2004/068242, Japanese Unexamined Patent Application, Publication No. 2005-173474, and Japanese Unexamined Patent Application, Publication No. 2006-48029). On the other hand, as radiation-sensitive acid generating agents, sulfonium salts having various functional groups have been disclosed (for example, see Japanese Unexamined Patent Application, Publication No. H03-148256).

### SUMMARY OF THE INVENTION

According to one aspect of the present invention, a radiation-sensitive resin composition includes a sulfonium compound represented by a following general formula (1), and a first polymer that serves as a base resin.

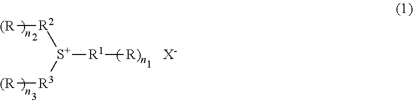

In the above general formula (1), R¹ represents an aromatic hydrocarbon group having a valency of (n₁+1) and 6 to 30 carbon atoms, an aliphatic chain hydrocarbon group having a valency of (n₁+1) and 1 to 10 carbon atoms or an alicyclic hydrocarbon group having a valency of (n₁+1) and 3 to 10 carbon atoms. R² represents an aromatic hydrocarbon group having a valency of (n₂+1) and 6 to 30 carbon atoms, an aliphatic chain hydrocarbon group having a valency of (n₂+1) and 1 to 20 carbon atoms or an alicyclic hydrocarbon group having a valency of (n₂+1) and 3 to 20 carbon atoms. R³ represents an aromatic hydrocarbon group is having a valency of (n₃+1) and 6 to 30 carbon atoms, an aliphatic chain hydrocarbon group having a valency of (n₃+1) and 1 to 30 carbon atoms or an alicyclic hydrocarbon group having a valency of (n₃+1) and 3 to 30 carbon atoms. Two among R¹ to R³ are optionally bonded with one another to form a cyclic structure including a sulfur cation. A part or all of hydrogen atoms R¹ to R³ have are unsubstituted or substituted. R represents a group represented by a following general formula (2). In a case where R is present in a plurality of number, Rs present in a plurality of number are each independent. n₁ to n₃ each independently represent an integer of 0 to 5, wherein n₁+n₂+n₃ 1. X⁻ represents an anion.

-A-R⁴ (2)

In the above general formula (2), R⁴ represents an alkali-dissociable group. A represents an oxygen atom, a —NR⁵— group, a —CO—O—* group or a —SO₂—O—* group. R⁵ represents a hydrogen atom or an alkali-dissociable group. “*” denotes a binding site to R⁴.

According to another aspect of the present invention, a method for forming a resist pattern includes forming a photoresist film on a substrate using the radiation-sensitive resin composition. The photoresist film is exposed. The exposed photoresist film is developed to form a resist pattern.

According to further aspect of the present invention, a sulfonium compound is represented by a following general formula (1).

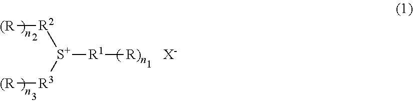

In the general formula (1), R² represents an aromatic hydrocarbon group having a valency of (n₁+1) and 6 to 30 carbon atoms, an aliphatic chain hydrocarbon group having a valency of (n₁+1) and 1 to 10 carbon atoms or an alicyclic hydrocarbon group having a valency of (n₁+1) and 3 to 10 carbon atoms. R² represents an aromatic hydrocarbon group having a valency of (n₂+1) and 6 to 30 carbon atoms, an aliphatic chain hydrocarbon group having a valency of (n₂+1) and 1 to 20 carbon atoms or an alicyclic hydrocarbon group having a valency of (n₂+1) and 3 to 20 carbon atoms. R³ represents an aromatic hydrocarbon group having a valency of (n₃+1) and 6 to 30 carbon atoms, an aliphatic chain hydrocarbon group having a valency of (n₃+1) and 1 to 30 carbon atoms, or an alicyclic hydrocarbon group having a valency of (n₃+1) and 3 to 30 carbon atoms. Two among R¹ to R³ are optionally bonded with one another to form a cyclic structure including a sulfur cation. A part or all of hydrogen atoms R¹ to R³ have are unsubstituted or substituted. R represents a group represented by a following general formula (2). In a case where R is present in a plurality of number, Rs present in a plurality of number are each independent. n₁ to n₃ each independently represent an integer is of 0 to 5, wherein n₁+n₂+n₃ 1. X⁻ represents an anion.

-A-R⁴ (2)

In the general formula (2), R⁴ represents an alkali-dissociable group. A represents an oxygen atom, a —NR⁵— group, a —CO—O—* group or a —SO₂—O—* group. R⁵ represents a hydrogen atom or an alkali-dissociable group. “*” denotes a binding site to R⁴.

### DESCRIPTION OF THE EMBODIMENTS

According to embodiments of the present invention, a radiation-sensitive resin composition, a method for forming a resist pattern and a sulfonium compound are provided as shown below.

A first aspect of the embodiments of the present invention provides a radiation-sensitive resin composition including (A) a sulfonium compound represented by the following general formula (1) (hereinafter, may be also referred to as “compound (A)”) and (B) a polymer that serves as a base resin (hereinafter, may be also referred to as “polymer (B)”).

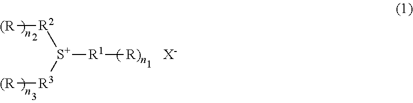

In the above general formula (1), R¹ represents an aromatic hydrocarbon group having a valency of (n₁+1) and 6 to 30 carbon atoms, an aliphatic chain hydrocarbon group having a valency of (n₁+1) and 1 to 10 carbon atoms or an alicyclic hydrocarbon group having a valency of (n₁+1) and 3 to 10 carbon atoms; R² represents an aromatic hydrocarbon group having a valency of (n₂+1) and 6 to 30 carbon atoms, an aliphatic chain hydrocarbon group having a valency of (n₂+1) and 1 to 20 carbon atoms or an alicyclic hydrocarbon group having a valency of (n₂+1) and 3 to 20 carbon atoms; R³ represents an aromatic hydrocarbon group having a valency of (n₃+1) and 6 to 30 carbon atoms, an aliphatic chain hydrocarbon group having a valency of (n₃+1) and 1 to 30 carbon atoms or an alicyclic hydrocarbon group having a valency of (n₃+1) and 3 to 30 carbon atoms, wherein, two among R¹ to R³ are optionally bonded with one another to form a cyclic structure including a sulfur cation, wherein a part or all of hydrogen atoms R¹ to R³ have are unsubstituted or substituted; R represents a group represented by the following general formula (2); provided that R is present in a plurality of number, Rs present in a plurality of number are each independent; n₁ to n₃ each independently represent an integer of 0 to 5, wherein n₁+n₂+n₃ 1; and X⁻ represents an anion.

-A-R⁴ (2)

In the above general formula (2), R⁴ represents an alkali-dissociable group; A represents an oxygen atom, a —NR⁵— group, a —CO—O—* group or a —SO₂—O—* group; wherein R⁵ represents a is hydrogen atom or an alkali-dissociable group; and “*” denotes a binding site to R⁴.)

A second aspect of the embodiments of the present invention provides the radiation-sensitive resin composition according to the first aspect including as the sulfonium compound (A) a compound represented by the following general formula (1-1).

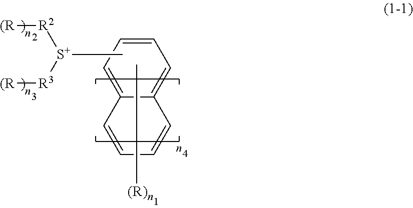

In the above general formula (1-1), R², R³, R, n₁ to n₃ and X⁻ are as defined in connection with the above general formula (1), wherein R² and R³ are optionally bonded with one another to form a cyclic structure including a sulfur cation; and n₄ represents 0 or 1.

A third aspect of the embodiments of the present invention provides the radiation-sensitive resin composition according to the first aspect including as the sulfonium compound (A) a compound represented by the following general formula (1-1a).

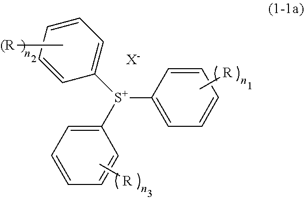

In the above general formula (1-1a), R, n₁ to n₃ and X⁻ are as defined in connection with the above general formula (1).

A fourth aspect of the embodiments of the present invention provides the radiation-sensitive resin composition according to any of the first to third aspects, wherein at least one R in the sulfonium compound (A) is a group represented by the following general formula (2a):

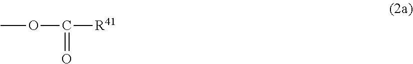

In the above general formula (2a), R⁴¹ represents a hydrocarbon group having 1 to 7 carbon atoms, wherein a part or all of hydrogen atoms are substituted with a fluorine atom, wherein provided that R represented by the above general formula (2a) is present in a plurality of number, R⁴¹s present in a plurality of number are each independent.

A fifth aspect of the embodiments of the present invention provides the radiation-sensitive resin composition according to any of the first to fourth aspects, further including (C) a polymer having a fluorine atom (hereinafter, may be also referred to as “polymer (C)”).

A sixth aspect of the embodiments of the present invention provides the radiation-sensitive resin composition according to the fifth aspect, wherein an amount of the polymer having a fluorine atom (C) blended is 0.1 to 20 parts by mass with respect to 100 parts by mass of the polymer (B).

A seventh aspect of the embodiments of the present invention provides a method for forming a resist pattern including the steps of (1) forming a photoresist film on a substrate using the radiation-sensitive resin composition according to any one of the first to sixth aspects, (2) exposing the photoresist film, and (3) developing the exposed photoresist film to form a resist pattern.

An eighth aspect of the embodiments of the present invention provides the method for forming a resist pattern according to the seventh aspect, wherein liquid immersion lithography of the photoresist film is carried out in the step (2).

A ninth aspect of the embodiments of the present invention provides a sulfonium compound represented by the following general formula (1):

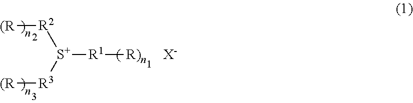

In the above general formula (1), R¹ represents an aromatic hydrocarbon group having a valency of (n₁+1) and 6 to 30 carbon atoms, an aliphatic chain hydrocarbon group having a valency of (n₁+1) and 1 to 10 carbon atoms or an alicyclic hydrocarbon group having a valency of (n₁+1) and 3 to 10 carbon atoms; R² represents an aromatic hydrocarbon group having a valency of (n₂+1) and 6 to 30 carbon atoms, an aliphatic chain hydrocarbon group having a valency of (n₂+1) and 1 to 20 carbon atoms or an alicyclic hydrocarbon group having a valency of (n₂+1) and 3 to 20 carbon atoms; R³ represents an aromatic hydrocarbon group having a valency of (n₃+1) and 6 to 30 carbon atoms, an aliphatic chain hydrocarbon group having a valency of (n₃+1) and 1 to 30 carbon atoms, or an alicyclic hydrocarbon group having a valency of (n₃+1) and 3 to 30 carbon atoms, wherein two among R¹ to R³ are optionally bonded with one another to form a cyclic structure including a sulfur cation, wherein a part or all of hydrogen atoms R¹ to R³ have are unsubstituted or substituted; R represents a group represented by the following general formula (2), wherein provided that R is present in a plurality of number, Rs present in a plurality of number are each independent; n₁ to n₃ each independently represent an integer of 0 to 5, wherein n₁+n₂+n₃ 1; and X⁻ represents an anion.

-A-R⁴ (2)

In the above general formula (2), R⁴ represents an alkali-dissociable group; A represents an oxygen atom, a —NR⁵— group, a —CO—O—* group or a —SO₂—O—* group; R⁵ represents a hydrogen atom or an alkali-dissociable group; and “*” denotes a binding site to R⁴.

A tenth aspect of the embodiments of the present invention provides the sulfonium compound according to the ninth aspect, the sulfonium compound being represented by the following general formula (1-1):

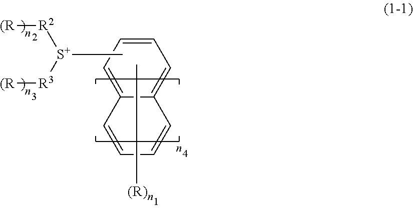

In the above general formula (1-1), R², R³, R, n₁ to n₃ and X⁻ are as defined in connection with the above general formula (1); wherein R² and R³ are optionally bonded with one another to form a cyclic structure including a sulfur cation; and n₄ represents 0 or 1.

An eleventh aspect of the embodiments of the present invention provides the sulfonium compound according to the ninth or tenth aspect, the sulfonium compound being represented by the following general formula (1-1a):

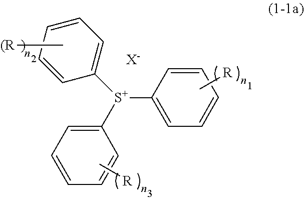

In the above general formula (1-1a), R, n₁ to n₃ and X⁻ are as defined in connection with the above general formula (1).

A twelfth aspect of the embodiments of the present invention provides the sulfonium compound according to any one of the ninth to eleventh aspects, wherein at least one R in the sulfonium compound (A) is a group represented by the following general formula (2a):

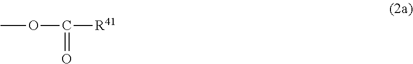

In the above general formula (2a), R⁴¹ represents a hydrocarbon group having 1 to 7 carbon atoms, wherein a part or all of hydrogen atoms are substituted with a fluorine atom, wherein provided that R represented by the above general formula (2a) is present in a plurality of number, R⁴¹s present in a plurality of number are each independent.

The radiation-sensitive resin composition of the embodiment of the present invention has effects that the radiation-sensitive resin composition is excellent in rectangularity in the cross-sectional shape of a resist pattern after development, less likely to cause scum and particularly less likely to cause development defects derived from an undissolved matter during development even for use of liquid immersion lithography. The defect is considered to result from aggregation of components in a photoresist film in a developer solution and reattachment on a pattern.

In addition, according to the method for forming a resist pattern of the embodiment of the present invention, effects are achieved that development defects are less likely to be caused and a pattern having a favorable shape can be efficiently formed.

Furthermore, the sulfonium compound of the embodiment of the present invention has effects that a radiation-sensitive resin composition can be produced which is excellent in rectangularity in the cross-sectional shape of a resist pattern obtained after development, less likely to cause scum and particularly less likely to cause development defects even for use of liquid immersion lithography.

Hereinafter, the preferred mode for carrying out the invention will be described. However, the present invention is not limited to the following preferred mode. Modification, improvement, and the like of the following preferred mode based on the ordinary skills of persons skilled in the art are included in the scope of the present invention within the range not to depart from the spirit of the present invention.

### I. Sulfonium Compound:

The sulfonium compound of the embodiment of the present invention is a component acting as an acid generating agent in the radiation-sensitive resin composition of the embodiment of the present invention described below, that is, a component that generates an acid at a light-exposed site when a photoresist film formed by the radiation-sensitive resin composition is exposed through a liquid for immersion lithography. The sulfonium compound is very different from compounds used for conventional acid generating agents in that the sulfonium compound has an alkali-dissociable group. The alkali-dissociable group reacts with an alkaline developer to form a polar group. It is considered that forming the polar group prevents the sulfonium compound from aggregation by a developer solution and a rinse solution, and defects derived from an undissolved matter from occurring.

1. Group Represented by the above General Formula (2):

The group represented by the above general formula (2) is a group derived by modifying a hydroxyl group, an amino group, a carboxyl group or a sulfoxyl group with an alkali-dissociable group. The group represented by the general formula (2) reacts with an alkali aqueous solution as shown in the reaction formula (3) to form a polar group -AH.

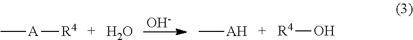

In the reaction formula (3), R⁴ represents an alkali-dissociable group. Herein, the term “alkali-dissociable group” refers to a group that substitutes for a hydrogen atom in a polar functional group and dissociates under basic conditions (for example, under a temperature condition at 23° C., by an action of a 2.38% by mass aqueous tetramethylammonium hydroxide solution).

The alkali-dissociable group is not particularly limited as long as the alkali-dissociable group shows the above-mentioned properties. It is to be noted that in the above general formula (2), preferred examples of the alkali-dissociable group in the case where A is an oxygen atom or a —NR⁵— group include a group represented by the following general formula (R⁴-1):

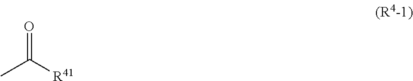

In the general formula (R⁴-1), R⁴¹ represents a hydrocarbon group having 1 to 7 carbon atoms wherein at least one hydrogen atom is substituted with a fluorine atom.

In the general formula (R⁴-1), preferred examples of R⁴¹ include linear or branched alkyl groups having 1 to 7 carbon atoms wherein at least one hydrogen atom is substituted with a fluorine atom, alicyclic hydrocarbon groups having 3 to 7 carbon atoms wherein a part or all of hydrogen atoms are substituted with fluorine atoms, and the like.

Specific examples of the linear or branched alkyl groups having 1 to 7 carbon atoms include a methyl group, an ethyl group, a 1-propyl group, a 2-propyl group, a 1-butyl group, a 2-butyl group, a 2-(2-methylpropyl) group, a 1-pentyl group, a 2-pentyl group, a 3-pentyl group, a 1-(2-methylbutyl) group, a 1-(3-methylbutyl) group, a 2-(2-methylbutyl) group, a 2-(3-methylbutyl) group, a neopentyl group, a 1-hexyl group, a t-hexyl group, a 3-hexyl group, a 1-(2-methylpentyl) group, a 1-(3-methylpentyl) group, a 1-(4-methylpentyl) group, a 2-(2-methylpentyl) group, a 2-(3-methylpentyl) group, a 2-(4-methylpentyl) group, a 3-(2-methylpentyl) group, a 3-(3-methylpentyl) group, and the like.

Specific examples of the alicyclic hydrocarbon groups having 3 to 7 carbon atoms include a cyclopentyl group, a cyclopentyl methyl group, a 1-(1-cyclopentylethyl) group, a 1-(2-cyclopentylethyl) group, a cyclohexyl group, a cyclohexylmethyl group, a cycloheptyl group, a 2-norbornyl group, and the like.

The group represented by R⁴¹ is a linear or branched alkyl group having 1 to 7 carbon atoms, and further preferably a group wherein: one of hydrogen atoms that carbon atoms which are connected to carbonyl group have is substituted with a fluorine atom; or hydrogen atoms that carbon atoms which are connected to carbonyl group have are not substituted, and all hydrogen atoms other carbon atoms have are substituted with a fluorine atom, and particularly preferably a 2,2,2-trifluoroethyl group.

In addition, in the above general formula (2), preferred examples of alkali-dissociable groups in the case where A is a —CO—O—* group include groups represented by the following general formulae (R⁴-2) to (R⁴-4):

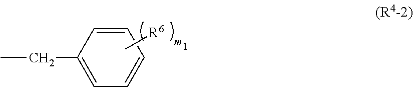

In the general formula (R⁴-2), m₁ represents an integer of 0 to 5; R⁶ represents a halogen atom, an alkyl group having 1 to 10 carbon atoms, an alkoxyl group having 1 to 10 carbon atoms, an acyl group having 2 to 10 carbon atoms or an acyloxy group having 2 to 10 carbon atoms; wherein provided that m₁ is 2 or more, R⁶s present in a plurality of number are each independent;

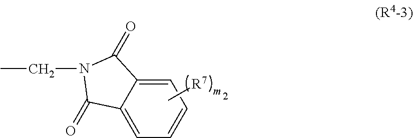

In the general formula (R⁴-3), m₂ represents an integer of 0 to 4; R⁷ represents a halogen atom, an alkyl group having 1 to 10 carbon atoms, an alkoxyl group having 1 to 10 carbon atoms, an acyl group having 2 to 10 carbon atoms, or an acyloxy group having 2 to 10 carbon atoms; wherein provided that m₂ is 2 or more, R⁷s present in a plurality of number are each independent;

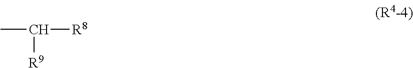

In the general formula (R⁴-4), R⁸ and R⁹ each independently represent a hydrogen atom or an alkyl group having 1 to 10 carbon atoms; wherein R⁸ and R⁹ are optionally bonded with one another to form an alicyclic structure having 4 to 20 carbon atoms.

Among groups represented by R⁶ in the above general formula (R⁴-2) and R⁷ in the above general formula (R⁴-3), examples of the halogen atom include a fluorine atom, a chlorine atom, a bromine atom, an iodine atom, and the like. Among them, a fluorine atom is preferable.

Examples of the alkyl group having 1 to 10 carbon atoms include a methyl group, an ethyl group, a 1-propyl group, a 2-propyl group, a 1-butyl group, a 2-butyl group, a 2-(2-methylpropyl) group, a 1-pentyl group, a 2-pentyl group, a 3-pentyl group, a 1-(2-methylbutyl) group, a 1-(3-methylbutyl) group, a 2-(2-methylbutyl) group, a 2-(3-methylbutyl) group, a neopentyl group, a 1-hexyl group, a 2-hexyl group, a 3-hexyl group, 1-(2-methylpentyl) group, a 1-(3-methylpentyl) group, a 1-(4-methylpentyl) group, a 2-(2-methylpentyl) group, a 2-(3-methylpentyl) group, a 2-(4-methylpentyl) group, a 3-(2-methylpentyl) group, a 3-(3-methylpentyl) group, and the like.

Examples of the alkoxyl group having 2 to 10 carbon atoms include a methoxy group, an ethoxy group, an n-butoxy group, a t-butoxy group, a propoxy group, an isopropoxy group, and the like. Examples of the acyl group having 2 to 10 carbon atoms include an acetyl group, an ethylcarbonyl group, a propylcarbonyl group, and the like. Examples of the acyloxy group having 2 to 10 carbon atoms include an acetoxy group, an ethyryloxy group, a butyryloxy group, a t-butyryloxy group, a t-amilyloxy group, an n-hexanecarbonyloxy group, an n-octanecarbonyloxy group, and the like.

Among groups represented by R⁸ and R⁹ in the above general formula (R⁴-4), examples of the alkyl group having 1 to 10 carbon atoms include the same alkyl groups having 1 to 10 carbon atoms as exemplified in R⁶ and R⁷.

Examples of the alicyclic structure having 4 to 20 carbon atoms formed with R⁸ and R⁹ which are bonded each other and carbon atoms to which each of R⁸ and R⁹ are bonded include a cyclopentyl group, a cyclopentylmethyl group, a 1-(1-cyclopentylethyl) group, a 1-(2-cyclopentylethyl) group, a cyclohexyl group, a cyclohexylmethyl group, a 1-(1-cyclohexylethyl) group, a 1-(2-cyclohexylethyl) group, a cycloheptyl group, a cycloheptylmethyl group, a 1-(1-cycloheptylethyl) group, a 1-(2-cycloheptylethyl) group, a 2-norbornyl group, and the like.

Specific examples of the group represented by the above is general formula (R⁴-4) include a methyl group, an ethyl group, a 1-propyl group, a 2-propyl group, a 1-butyl group, a 2-butyl group, a 1-pentyl group, a 2-pentyl group, a 3-pentyl group, a 1-(2-methylbutyl) group, a 1-(3-methylbutyl) group, a 2-(3-methylbutyl) group, a neopentyl group, a 1-hexyl group, a 2-hexyl group, a 3-hexyl group, a 1-(2-methylpentyl) group, a 1-(3-methylpentyl) group, a 1-(4-methylpentyl) group, a 2-(3-methylpentyl) group, a 2-(4-methylpentyl) group, a 3-(2-methylpentyl) group, and the like. Among them, a methyl group, an ethyl group, a 1-propyl group, a 2-propyl group, a 1-butyl group and a 2-butyl group are preferable.

The group represented by the above general formula (2) can be formed by fluoroacylating functional groups such as a hydroxyl group, an amino group, a carboxyl group by a conventionally well-known method. More specifically, examples of the method include (1) esterification by condensation of an alcohol and a fluorocarboxylic acid in the presence of an acid, (2) esterification by condensation of an alcohol and a fluorocarboxylic halide in the presence of a base.

### 2. Compound Represented by the General Formula (1):

Among groups represented by R¹ to R³ in the above general formula (1), examples of the aromatic hydrocarbon group having 6 to 30 carbon atoms include groups derived from benzene, naphthalene, and the like. Examples of the aliphatic chain hydrocarbon group include groups derived from linear or branched alkyl groups such as methane, ethane, n-butane, 2-methylpropane, 1-methylpropane, tert-butane, n-pentane, and the like. Examples of the alicyclic hydrocarbon group include groups derived from alicyclic hydrocarbons such as cyclobutane, cyclopentane, cyclohexane, cyclooctane, a norbornyl group, tricyclodecane, tetracyclododecane, adamantine, and the like.

In the case where two among R¹ to R³ are bonded with one another to form a cyclic structure including a sulfur cation, the cyclic structure is preferably a 5-membered ring or 6-membered ring, and more preferably a 5-membered ring (i.e., a tetrahydrothiophene ring).

In the above general formula (1), examples of the substituent that may substitute for a part or all of hydrogen atoms that R¹ to R³ have include a halogen atom, a linear or branched alkyl group having 1 to 10 carbon atoms, a linear or branched alkoxyl group having 1 to 10 carbon atoms, a linear or branched alkoxycarbonyl group having 2 to 11 carbon atoms, a linear, branched or cyclic alkanesulfonyl group having 1 to 10 carbon atoms, a hydroxyl group, an alkoxyalkyl group, an alkoxycarbonyloxy group, a carboxyl group, a cyano group, a nitro group, and the like.

Examples of the linear or branched alkyl group having 1 to 10 carbon atoms include a methyl group, an ethyl group, an n-butyl group, a 2-methylpropyl group, a 1-methylpropyl group, a tert-butyl group, an n-pentyl group, and the like. Among them, a methyl group, an ethyl group, an n-butyl group and a tert-butyl group are preferable.

Examples of the linear or branched alkoxyl group having 1 to 10 carbon atoms include a methoxy group, an ethoxy group, an n-propoxy group, an isopropoxy group, an n-butoxy group, a 2-methylpropoxy group, a 1-methylpropoxy group, a tert-butoxy group, and the like. Among them, a methoxy group, an ethoxy group, an n-propoxy group and an n-butoxy group are preferable.

Examples of the linear or branched alkoxycarbonyl group having 2 to 11 carbon atoms include a methoxycarbonyl group, an ethoxycarbonyl group, an n-propoxycarbonyl group, an isopropoxycarbonyl group, an n-butoxycarbonyl group, a 2-methylpropoxycarbonyl group, a 1-methylpropoxycarbonyl group, a tert-butoxycarbonyl group, and the like. Among them, a methoxycarbonyl group, an ethoxycarbonyl group, an n-butoxycarbonyl group are preferable.

Examples of the linear, branched or cyclic alkanesulfonyl group having 1 to 10 carbon atoms include a methanesulfonyl group, an ethanesulfonyl group, an n-propane sulfonyl group, an n-butanesulfonyl group, a tert-butanesulfonyl group, a cyclopentanesulfonyl group, a cyclohexanesulfonyl group, and the like. Among them, a methanesulfonyl group, an ethanesulfonyl group, an n-propane sulfonyl group, an n-butanesulfonyl group, a cyclopentanesulfonyl group and a cyclohexanesulfonyl group are preferable.

Examples of the alkoxyalkyl group include a linear, branched or cyclic alkoxyalkyl group having 2 to 21 carbon atoms such as a methoxymethyl group, an ethoxymethyl group, a 1-methoxyethyl group, a 2-methoxyethyl group, a 1-ethoxyethyl group, a 2-ethoxyethyl group, and the like.

Examples of the alkoxycarbonyloxy group include linear, branched or cyclic alkoxycarbonyloxy groups having 2 to 21 carbon atoms such as a methoxycarbonyloxy group, an ethoxycarbonyloxy group, an n-propoxycarbonyloxy group, an isopropoxycarbonyloxy group, an n-butoxycarbonyloxy group, a tert-butoxycarbonyloxy group, a cyclopentyloxycarbonyloxy group, a cyclohexyloxycarbonyloxy, and the like.

Among them, the sulfonium compound is preferably a compound represented by the above general formula (1-1) wherein R¹ is a phenyl group or a naphthyl group, and more preferably a compound represented by the above general formula (1-1a) wherein R¹ to R³ are a phenyl group.

It is to be noted that the sulfonium compound may be contained either alone or in combination of two or more types thereof.

In the general formula (1), X⁻ is an anion. Specific is examples of the anion include anions represented by the following general formulae (4), (5), (6-1), (6-2), and the like.

R¹⁰CₚF₂ₚSO₃⁻ (4)

In the general formula (4), R¹⁰ represents a hydrogen atom, a fluorine atom or a hydrocarbon group having 1 to 12 carbon atoms; and p is an integer of 1 to 10.

R¹¹SO₃⁻ (5)

In the general formula (5), R¹¹ represents a hydrogen atom, a fluorine atom or a hydrocarbon group having 1 to 12 carbon atoms.

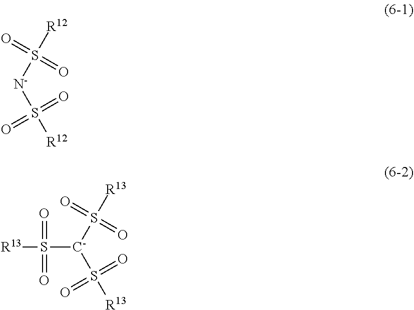

In the general formula (6-1), R¹²s each independently represent a linear or branched aliphatic hydrocarbon group having 1 to 10 carbon atoms and a fluorine atom; wherein two R¹²s are bonded with one another to form a cyclic structure having 5 to 10 membered rings and fluorine atoms; in addition, in the general formula (6-2), R¹³s each independently represent a linear or branched aliphatic hydrocarbon group having 1 to 10 carbon atoms and a fluorine atom; wherein any two of R¹³ are optionally bonded with one another to form a cyclic structure having 5 to 10 cycle members and fluorine atoms.

In the case where X⁻ is an anion represented by the general formula (4), “—CₚF₂ₚ—” is a perfluoroalkylene group having p carbon atoms. The group may be linear or branched. It is to be noted that p is preferably 1, 2, 4 or 8.

In the case where R¹⁰ in the general formula (4) and R¹¹ in the general formula (5) are a hydrocarbon group having 1 to 12 carbon atoms, the hydrocarbon group may be an unsubstituted hydrocarbon group (i.e., an alkyl group, a cycloalkyl group, a bridged alicyclic hydrocarbon group, or the like) or a hydrocarbon group derived by substituting hydrogen atom(s) in the hydrocarbon groups with at least one kind of substituents of a hydroxyl group, a carboxyl group, a cyano group, a nitro group, an alkoxyl group, an alkoxyalkyl group, an alkoxycarbonyl group, an alkoxycarbonyloxy group, and the like. Preferred examples of the hydrocarbon groups include a methyl group, an ethyl group, an n-propyl group, an isopropyl group, an n-butyl group, a 2-ethylhexyl group, an n-nonyl group, an n-decyl group, a norbornyl group, a norbornylmethyl group, a hydroxynorbornyl group and an adamantyl group.

In the case where R¹² in the general formula (6-1) and R¹³ in the general formula (6-2) are a linear or branched aliphatic hydrocarbon group having 1 to 10 carbon atoms with fluorine atoms, specific examples of the aliphatic hydrocarbon group include a trifluoromethyl group, a pentafluoroethyl group, a heptafluoropropyl group, a nonafluorobutyl group, a dodecafluoropentyl group, a perfluorooctyl group, and the like. In the case where two R¹²s in the general formula (6-1) and any two of R¹³s in the general formula (6-2) are optionally bonded with one another to form a bivalent 5- to 10-membered group having fluorine atoms, specific examples of the bivalent group include a tetrafluoroethylene group, a hexafluoropropylene group, an octafluorobutylene group, a decafluoropentylene group, an undecafluorohexylene group, and the like. It is to be noted that the bivalent group may have a substituent.

Preferred examples of X⁻ include trifluoromethanesulfonate anion, perfluoro-n-butanesulfonate anion, perfluoro-n-octanesulfonate anion, 2-bicyclo[2.2.1]hept-2-yl-1,1,2,2-tetrafluoroethanesulfonate anion, 2-bicyclo[2.2.1]hept-2-yl-1,1-difluoroethanesulfonate anion, 1,1-difluoro-2-(1-adamantyl)ethane-1-sulfonate anion, 6-(1-adamantanecarbonyloxy)-1,1,2,2-tetrafluorohexane-1-sulfonate anion, anions represented by the following formulae (7-1) to (7-7).

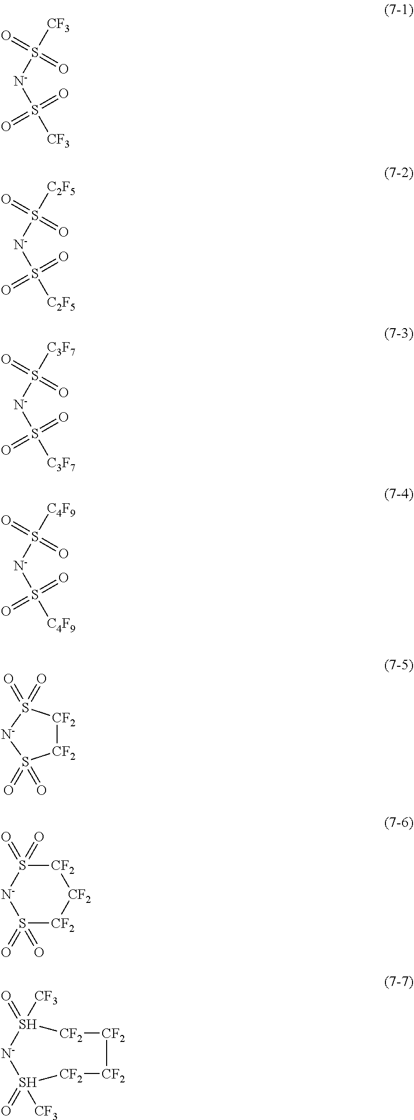

The sulfonium compound represented by the general formula (1) can be synthesized by a method for the production including (A) a step of reacting, for example, a compound represented by the following formula (1a) with a compound represented by the following formula (1b) or a derivative derived from the compound to obtain a compound represented by the following formula (1c), and (B) a step of reacting the obtained compound represented by the following formula (1c) with a compound represented by the following formula (1d) to obtain the compound represented by the general formula (1). It is to be noted that examples of the derivative of the compound represented by the following formula (1b) include carboxylic acid ester compounds, carboxylic anhydrides, and the like, for example, in the case where the compound represented by the following formula (1b) is a carboxylic acid.

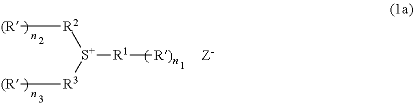

In the general formula (1a), R¹, R², R³, and n₁ to n₃ are as defined in connection with the above general formula (1), and R′ represents a group represented by the following general formula (2a′), provided that R′ is present in a plurality of number, R′s in a plurality of number are each independent; and Z⁻ represents a halogen atom,

-A-H (2a)

In the general formula (2a′), A is as defined in connection with the above general formula (2),

R⁴—OH (1b)

In the general formula (1b), R⁴ is as defined in connection with the above general formula (2),

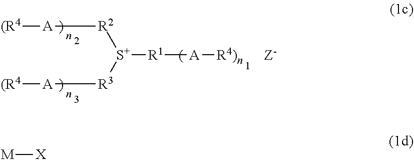

In the general formula (1d), M represents an alkali metal; X is as defined in connection with the above general formula (1).

The conditions of the step A are not particularly limited and may involve a reaction temperature of typically −30 to 100° C., preferably −20 to 90° C., and particularly preferably −10 to 80° C.; and a reaction time of typically 0.1 to 48 hrs, preferably 0.5 to 24 hrs, and particularly preferably 1 to 10 hrs.

In addition, in the step A, an organic solvent such as methylene chloride, chloroform, carbon tetrachloride and/or 1,2-dichloroethane, as well as water may be used as a solvent. The amount of the solvent used is typically 0.1 to 50 g, preferably 1 to 30 g and particularly preferably 2 to 20 g per gram of the compound (3).

In the step A, the molar ratio of a compound represented by the above formula (1b) to a compound represented by the above formula (1a) or a derivative derived from the compound (i.e., a value: the compound represented by the above formula (1b) or a derivative therefrom/the compound represented by the above formula (1a)) is typically 0.5 to 20 and preferably 1 to 10.

The conditions of the step B are not particularly limited and may involve a reaction temperature of typically −30 to 100° C., preferably −20 to 90° C., and particularly preferably −10 to 80° C.; and a reaction time of typically 0.1 to 48 hrs, preferably 0.5 to 24 hrs, and particularly preferably 1 to 10 hrs.

In the step B, the molar ratio of the compound represented by the above formula (1d) to the compound represented by the above formula (1c) (i.e., a value: the compound represented by the above formula (1d) or a derivative derived from the compound/the compound represented by the above formula (1c)) is typically 0.1 to 20 and preferably 0.5 to 5.

### II. Radiation-Sensitive Resin Composition:

The radiation-sensitive resin composition of the embodiment of the present invention contains the compound (A) as an acid generating agent and the polymer (B). The radiation-sensitive resin composition of the embodiment of the present invention is excellent in rectangularity in the cross-sectional shape of a resist pattern obtained after development and less likely to cause scum. Particularly, since the compound (A) has an alkali-dissociable group, aggregation by a developer solution and a rinse solution can be prevented, whereby a main reason of defects is considered to be minimized. Therefore, the radiation-sensitive resin composition of the embodiment of the present invention has a superior effect that development defects are less likely to be caused particularly in use for liquid immersion lithography.

### 1. Acid Generating Agent:

An acid generating agent generates an acid by irradiation with a radioactive ray at a light-exposed site. The radiation-sensitive resin composition of the embodiment of the present invention contains the compound (A) as an acid generating agent. It is to be noted that the radiation-sensitive resin composition of the embodiment of the present invention may contain the compound (A) either alone or in combination with other acid generating agent (hereinafter, may be also referred to as “other acid generating agent”) as an acid generating agent.

Examples of the other acid generating agent include an onium salt compound, a sulfone compound, a sulfonic acid ester compound, a sulfonimide compound, a diazomethane compound, a disulfonylmethane compound, an oximesulfonate compound, a hydrazinesulfonate compound, and the like. Among them, at least one selected from the group consisting of an onium salt compound, a sulfonimide compound, and a diazomethane compound is preferable.

Examples of the other acid generating agent include the compound described in paragraphs nos. 0086 to 0113 of PCT International Publication No. 2009/051088.

Particularly preferred specific examples of the other acid generating agent include triphenylsulfonium trifluoromethanesulfonate, triphenylsulfonium nonafluoro-n-butanesulfonate, triphenylsulfonium p-toluenesulfonate, triphenylsulfonium 10-camphorsulfonate, triphenylsulfonium 2-trifluoromethylbenzenesulfonate, triphenylsulfonium 4-trifluoromethylbenzenesulfonate, triphenylsulfonium 2,4-difluorobenzenesulfonate, triphenylsulfonium 1,1,2,2-tetrafluoro-2-(norbornan-2-yl)ethanesulfonate, triphenylsulfonium 2-(5-t-butoxycarbonyloxybicyclo[2.2.1]heptan-2-yl)-1,1,2,2-tetrafluoroethanesulfonate, triphenylsulfonium 2-(6-t-butoxycarbonyloxybicyclo[2.2.1]heptan-2-yl)-1,1,2,2-tetrafluoroethanesulfonate, triphenylsulfonium 2-(5-pivaloyloxybicyclo[2.2.1]heptan-2-yl)-1,1,2,2-tetrafluoroethanesulfonate, triphenylsulfonium 2-(6-pivaloyloxybicyclo[2.2.1]heptan-2-yl)-1,1,2,2-tetrafluoroethanesulfonate, triphenylsulfonium 2-(5-hydroxybicyclo[2.2.1]heptan-2-yl)-1,1,2,2-tetrafluoroethanesulfonate, triphenylsulfonium 2-(6-hydroxybicyclo[2.2.1]heptan-2-yl)-1,1,2,2-tetrafluoroethanesulfonate,

triphenylsulfonium 2-(5-methanesulfonyloxybicyclo[2.2.1]heptan-2-yl)-1,1,2,2-tetrafluoroethanesulfonate, triphenylsulfonium 2-(6-methanesulfonyloxybicyclo[2.2.1]heptan-2-yl)-1,1,2,2-tetrafluoroethanesulfonate, triphenylsulfonium 2-(5-1-propanesulfonyloxybicyclo[2.2.1]heptan-2-yl)-1,1,2,2-tetrafluoroethanesulfonate, triphenylsulfonium 2-(6-1-propanesulfonyloxybicyclo[2.2.1]heptan-2-yl)-1,1,2,2-tetrafluoroethanesulfonate, triphenylsulfonium 2-(5-n-hexanesulfonyloxybicyclo[2.2.1]heptan-2-yl)-1,1,2,2-tetrafluoroethanesulfonate, triphenylsulfonium 2-(6-n-hexanesulfonyloxybicyclo[2.2.1]heptan-2-yl)-1,1,2,2-tetrafluoroethanesulfonate, triphenylsulfonium 2-(5-oxobicyclo[2.2.1]heptan-2-yl)-1,1,2,2-tetrafluoroethanesulfonate, triphenylsulfonium 2-(6-oxobicyclo[2.2.1]heptan-2-yl)-1,1,2,2-tetrafluoroethanesulfonate, triphenylsulfonium 1,1-difluoro-2-(bicyclo[2.2.1]heptan-2-yl)ethanesulfonate,

1-(4-n-butoxynaphthalen-1-yl)tetrahydrothiophenium trifluoromethanesulfonate, 1-(4-n-butoxynaphthalen-1-yl)tetrahydrothiophenium nonafluoro-n-butanesulfonate, 1-(4-n-butoxynaphthalen-1-yl)tetrahydrothiophenium 1,1,2,2-tetrafluoro-2-(norbornan-2-yl)ethanesulfonate, 1-(4-n-butoxynaphthalen-1-yl)tetrahydrothiophenium 2-(5-t-butoxycarbonyloxybicyclo[2.2.1]heptan-2-yl)-1,1,2,2-tetrafluoroethanesulfonate, 1-(4-n-butoxynaphthalen-1-yl)tetrahydrothiophenium 2-(6-t-butoxycarbonyloxybicyclo[2.2.1]heptan-2-yl)-1,1,2,2-tetrafluoroethanesulfonate, 1-(4-n-butoxynaphthalen-1-yl)tetrahydrothiophenium 1,1-difluoro-2-(bicyclo[2.2.1]heptan-2-yl)ethanesulfonate, and the like.

The proportion of the other acid generating agent used can be appropriately selected depending on its type. The proportion is typically no greater than 95 parts by mass, preferably no greater than 90 parts by mass and more preferably no greater than 80 parts by mass with respect to 100 parts by mass of the total of the compound (A) and other acid generating agents. When the proportion of the other acid generating agent used is in excess, desired effects of the embodiment of the present invention are likely to be diminished.

The amount of the acid generating agent blended may be variously selected depending on characteristics of resists. In the case where the radiation-sensitive resin composition is a positive type radiation-sensitive resin composition, the amount of the acid generating agent blended is preferably 0.001 to 70 parts by mass, more preferably 0.01 to 50 parts by mass and particularly preferably 0.1 to 20 parts by mass with respect to 100 parts by mass of the polymer (B). When the amount of the acid generating agent blended is no less than 0.001 parts by mass, decreases in sensitivity and resolution can be suppressed. On the other hand, when the amount of the acid generating agent blended is no greater than 70 parts by mass, decreases in coating property of resists and pattern configuration can be suppressed.

In addition, in the case where the radiation-sensitive resin composition is a negative type radiation-sensitive resin composition, the amount of the acid generating agent blended is preferably 0.01 to 70 parts by mass, more preferably 0.1 to 50 parts by mass and particularly preferably 0.5 to 20 parts by mass with respect to 100 parts by mass of the polymer (B). When the amount of the acid generating agent blended is less than 0.01 parts by mass, there is a tendency that sensitivity and resolution decrease. On the other hand, when the amount of the acid generating agent blended is more than 70 parts by mass, there is a tendency that deterioration in coating properties of resists and pattern configuration is likely to occur.

### 2. Polymer (B):

The polymer (B) is exemplified by a polymer which is insoluble or hardly soluble in alkali and has an acid-dissociable group and which becomes readily soluble in alkali when an acid-dissociable group dissociates (hereinafter, may be also referred to as “polymer (B1)”), and a polymer which is soluble in an alkaline developer and has one type or more functional groups having an affinity with an alkaline developer (hereinafter, may be also referred to as “polymer (B2)”). Examples of the functional groups having an affinity with an alkaline developer include functional groups containing an oxygen atom such as a phenolic hydroxyl group, an alcoholic hydroxyl group, a carboxyl group, and the like. The polymer (B1) can be suitably used as a base resin of the positive type radiation-sensitive resin composition. In addition, the polymer (B2) can be suitably used as a base resin of the negative type radiation-sensitive resin composition.

Herein, “insoluble or hardly soluble in alkali” refers to a property that no less than 50% of the initial film thickness of a coating remains in the case where the coating formed using only the polymer (B1) is developed in place of a photoresist film under alkali development conditions employed in forming a resist pattern from a photoresist film formed using a radiation-sensitive resin composition containing the polymer (B1).

In use together with (C) a polymer described below, the proportion of the fluorine atom(s) contained in the polymer (B) is preferably smaller than the proportion of the fluorine atom(s) contained in the polymer (C). In such a case, water repellency of the surface of a photoresist film formed from a radiation-sensitive resin composition containing the polymer (B) and the polymer (C) described later can be enhanced, thereby eliminating necessity of separately forming an upper layer film upon liquid immersion lithography. The proportion of the fluorine atom(s) contained in the polymer (B) is typically less than 10% by mass, preferably 0 to 9% by mass and more preferably 0 to 6% by mass with respect to 100% by mass of the total of the polymer (B). It is to be noted that the proportion of the fluorine atom(s) contained in the polymer (B) can be determined by ¹³C-NMR.

(Polymer (B1))

The acid-dissociable group in the polymer (B1) refers to a group which is derived by substituting hydrogen atom(s) in acidic functional groups such as, for example, a phenolic hydroxyl group, a carboxyl group and sulfonic acid, and which dissociates in the presence of an acid. Examples of such acid-dissociable groups include a substituted methyl group, a 1-substituted ethyl group, a 1-substituted n-propyl group, a 1-branched alkyl group, an alkoxycarbonyl group, an acyl group, a cyclic acid-dissociable group, and the like.

Examples of the substituted methyl group include a group described in paragraph no. 0117 of PCT International Publication No. 2009/051088. Examples of the 1-substituted ethyl group include a group described in paragraph no. 0118 of PCT International Publication No. 2009/051088. Examples of the 1-substituted n-propyl group include a group described in paragraph no. 0119 of PCT International Publication No. 2009/051088. Examples of the acyl group include a group described in paragraph no. 0120 of PCT International Publication No. 2009/051088. Examples of the cyclic acid-dissociable group include a group described in paragraph no. 0121 of PCT International Publication No. 2009/051088.

Among the acid-dissociable groups, a benzyl group, a t-butoxycarbonyl methyl group, a 1-methoxyethyl group, a 1-ethoxyethyl group, a 1-cyclohexyloxyethyl group, a 1-ethoxy-n-propyl group, a t-butyl group, a 1,1-dimethylpropyl group, a t-butoxycarbonyl group, a tetrahydropyranyl group, a tetrahydrofuranyl group, a tetrahydrothiopyranyl group, a tetrahydrothiofuranyl group, and the like are preferable. It is to be noted that two types or more of the acid-dissociable groups may exist in the polymer (B1).

The proportion of introduction of the acid-dissociable group in the polymer (B1) (rate of number of acid-dissociable group(s) to the total number of acidic functional group(s) and acid-dissociable group(s) in the polymer (B1)) can be appropriately selected depending on the type of the acid-dissociable group and the polymer (B1), and is preferably 5 to 100 mol % and more preferably 10 to 100 mol %.

The polymer (B1) is not particularly limited as long as the polymer (B1) has properties described above. Suitable examples of the polymer (B1) include polymers derived by substituting at least one of hydrogen atoms in phenolic hydroxyl group(s) in poly(4-hydroxystyrene) with an acid-dissociable group, polymers derived by substituting with an acid-dissociable group at least one of hydrogen atoms in phenolic hydroxyl group(s) and/or at least one of hydrogen atoms in carboxyl group(s) in a copolymer of 4-hydroxystyrene and/or 4-hydroxy-α-methylstyrene with (meth)acrylic acid, and the like. It is to be noted that the polymer (B1) may be used either alone or as a mixture of two or more thereof.

In addition, the polymer (B1) can be variously chosen depending on the type of radiation source used. For example, in the case where a KrF excimer laser is used as a radiation source, the polymer (B1) is preferably a polymer (hereinafter, may be also referred to as “polymer (KrF)”) which is insoluble or hardly soluble in alkali and has a repeating unit represented by the following general formula (8) (hereinafter, may be also referred to as “repeating unit (8)”) and a repeating unit derived by protecting phenolic hydroxyl group(s) in the repeating unit (8) with an acid-dissociable group. It is to be noted that the polymer (KrF) can be used in the case where other radiation source of an ArF excimer laser, an F₂ excimer laser, an electron beam, or the like is used.

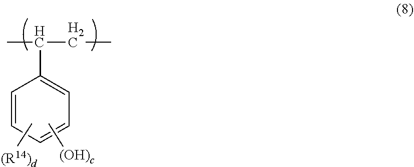

In the general formula (8), c and d each represent an integer of 1 to 3, wherein c+d≦5; and R¹⁴ represents a hydrogen atom or a monovalent organic group, wherein provided that R′4 is present in a plurality of number, R¹⁴s present in a plurality of number are each independent.

The repeating unit (8) is particularly preferably a repeating unit derived by cleaving a non-aromatic double bond in 4-hydroxystyrene. In addition, the polymer (KrF) may have other repeating units than the repeating unit (8).

Examples of the other repeating unit include a repeating unit in which a polymerizable unsaturated bond of vinyl aromatic compounds such as styrene, α-methylstyrene; (meth)acrylic acid esters such as t-butyl (meth)acrylate, adamantyl (meth)acrylate, 2-methyladamantyl (meth)acrylate is cleaved.

In the case where an ArF excimer laser is used as a radiation source, the polymer (B1) is particularly preferably a polymer (hereinafter, may be also referred to as “polymer (ArF)”) which is insoluble or hardly soluble in alkali and has a repeating unit represented by the following general formula (9) (hereinafter, may be also referred to as “repeating unit (9)”), and/or a repeating unit represented by the following general formula (10) (hereinafter, may be also referred to as “repeating unit (10)”), and a repeating unit represented by the following general formula (11) (hereinafter, may be also referred to as “repeating unit (11)”). It is to be noted that the polymer (ArF) can be used also in the case where other radiation source of a KrF excimer laser, an F₂ excimer laser, an electron beam or the like is used.

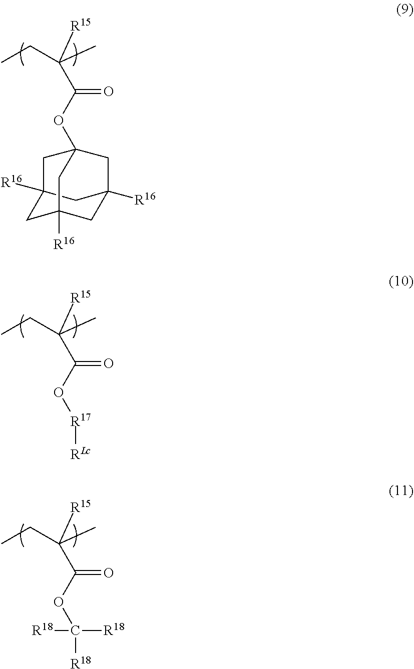

In the general formulae (9) to (11), R¹⁵ represents a hydrogen atom, a methyl group or a trifluoromethyl group.
In the general formula (9), a plurality of R¹⁶s each independently represent a hydrogen atom, a hydroxyl group, a cyano group or a —COOR¹⁹ group; and R¹⁹ represents a hydrogen atom, a linear or branched alkyl group having 1 to 4 carbon atoms, or a cycloalkyl group having 3 to 20 carbon atoms.

In the general formula (10), R¹⁷ represents a single bond, an ether group, an ester group, a carbonyl group, a bivalent aliphatic chain hydrocarbon group having 1 to 30 carbon atoms, a bivalent alicyclic hydrocarbon group having 3 to 30 carbon atoms, a bivalent aromatic hydrocarbon group having 6 to 30 carbon atoms, or a bivalent group provided by combining the same; and R^{Lc} represents a monovalent organic group having a lactone structure.

In the general formula (11), a plurality of R¹⁸s each independently represent a monovalent alicyclic hydrocarbon group having 4 to 20 carbon atoms or a derivative thereof, or a linear or branched alkyl group having 1 to 4 carbon atoms, wherein at least one R¹⁸ is an alicyclic hydrocarbon group or a derivative thereof, and any two R¹⁸s are optionally bonded with one another to form a bivalent alicyclic hydrocarbon group having 4 to 20 carbon atoms or a derivative thereof together with the carbon atom to which R¹⁸s are attached.

Suitable examples of the repeating unit (9) include those derived from 3-hydroxyadamantan-1-yl (meth)acrylate, 3,5-dihydroxyadamantan-1-yl (meth)acrylate, 3-cyano adamantan-1-yl (meth)acrylate, 3-carboxyadamantan-1-yl (meth)acrylate, 3,5-dicarboxyadamantan-1-yl (meth)acrylate, 3-carboxy-5-hydroxyadamantan-1-yl (meth)acrylate, 3-methoxycarbonyl-5-hydroxyadamantan-1-yl (meth)acrylate, and the like.

In the general formula (10), specific examples of the monovalent organic group having a lactone structure represented by R^{Lc} include groups represented by the following general formulae (R^{Lc}-1) to (R^{Lc}-6).

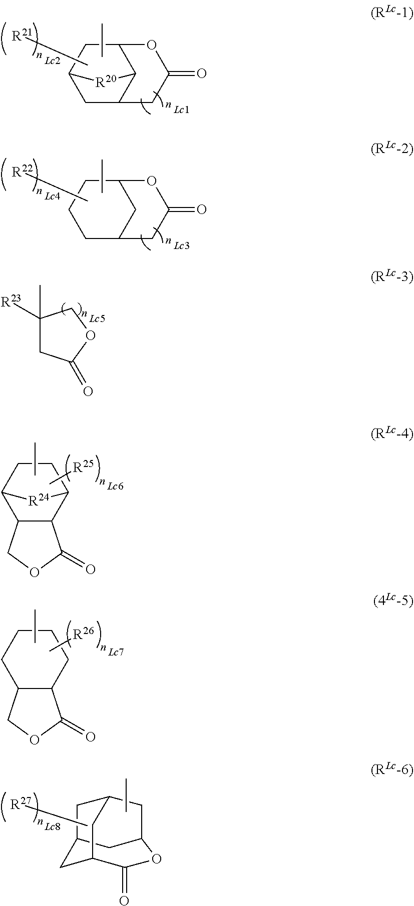

R²⁰ in the general formula (R^{Lc}-1) and R²⁴ in the general formula (R^{Lc}-4) represent an oxygen atom or a methylene group; R²¹ in (R^{Lc}-1), R²² in (R^{Lc}-2), R²³ in (R^{Lc}-3), R²⁵ in (R^{Lc}-5), R²⁶ in (R^{Lc}-5), and R²⁷ in (R^{Lc}-6) represent a hydrogen atom, a linear or branched alkyl group having 1 to 4 carbon atoms, a linear or branched fluorinated alkyl group having 1 to 4 carbon atoms, or a linear or branched alkoxyl group having 1 to 4 carbon atoms, wherein provided that any of R²⁰-R²⁷ is present in a plurality of number, the any of R²⁰-R²⁷s present in a plurality of number are each independent; n_{Lc1} in the general formula (R^{Lc}-1) and n_{Lc3} in the general formula (R^{Lc}-2) represent 0 or 1; n_{Lc2} in (R^{Lc}-2) represents an integer of 0 to 3; n_{Lc4} in (R^{Lc}-2) represents an integer of 0 to 6; n_{Lc5} in (R^{Lc}-3) represents an integer of 1 to 3; n_{Lc6} in (R^{Lc}-4) represents an integer of 0 to 2; n_{Lc7} in (R^{Lc}-5) represents an integer of 0 to 4; and n_{Lc8} in (R^{Lc}-6) represents an integer of 0 to 9.

Suitable examples of the repeating unit (11) include repeating units derived from 1-methylcyclopentyl (meth)acrylate, 1-ethylcyclopentyl (meth)acrylate, 1-methylcyclohexyl (meth)acrylate, 1-ethylcyclohexyl (meth)acrylate, 2-methyladamantan-2-yl (meth)acrylate, 2-ethyladamantan-2-yl (meth)acrylate, 2-n-propyladamantan-2-yl (meth)acrylate, 2-i-propyladamantan-2-yl (meth)acrylate, 1-(adamantan-1-yl)-1-methylethyl (meth)acrylate, and the like.

The polymer (ArF) can also have other repeating unit except for the repeating units (9) to (11). Examples of a monomer that provides the other repeating unit include mono-functional monomers such as: (meth)acrylic acid esters such as 7-oxo-6-oxabicyclo[3.2.1]octan-4-yl (meth)acrylate, 2-oxotetrahydropyran-4-yl (meth)acrylate, 4-methyl-2-oxotetrahydropyran-4-yl (meth)acrylate, 5-oxotetrahydrofuran-3-yl (meth)acrylate, 2-oxotetrahydrofuran-3-yl (meth)acrylate, (5-oxotetrahydrofuran-2-yl)methyl (meth)acrylate, (3,3-dimethyl-5-oxotetrahydrofuran-2-yl)methyl (meth)acrylate, and 2-hydroxyethyl (meth)acrylate; unsaturated amide compounds such as (meth)acrylamide, N,N-dimethyl(meth)acrylamide, crotonamide, maleinamide, fumaramide, mesacondiamide, citraconamide, and itaconamide; unsaturated polycarboxylic acid anhydrides such as maleic anhydride, and itaconic anhydride; bicyclo[2.2.1]hept-2-ene or a derivative thereof; and tetracyclo[6.2.1.1^{3,6}0^{2,7}]dodec-3-ene or a derivative thereof, and poly-functional monomers such as methylene glycol di(meth)acrylate, ethylene glycol di(meth)acrylate, 2,5-dimethyl-2,5-hexanediol di(meth)acrylate, 1,2-adamantane diol di(meth)acrylate, 1,3-adamantane diol di(meth)acrylate, 1,4-adamantane diol di(meth)acrylate, and tricyclodecanedimethylol di(meth)acrylate.

Further, in the case where an F₂ excimer laser is used as a radiation source, specific examples of the polymer (B1) include the polymer described in paragraph no. 0136 to paragraph no. 0147 of PCT International Publication No. 2009/051088.

A preparation method of the polymer (B1) is not particularly limited. Examples of the method include a method for introducing one or more types of acid-dissociable group(s) into an acidic functional group in an alkali-soluble polymer prepared in advance; a method for polymerizing one or more types of polymerizable unsaturated monomer(s) having an acid-dissociable group with one or more types of other polymerizable unsaturated monomer(s) as needed; a method for polycondensing one or more types of polycondensable component(s) having an acid-dissociable group with other polycondensable component(s) as needed, and the like.

Polymerization of a polymerizable unsaturated monomer upon preparation of an alkali-soluble polymer and polymerization of a polymerizable unsaturated monomer having an acid-dissociable group can be carried out in appropriate polymerization systems for e.g., bulk polymerization, solution polymerization, precipitation polymerization, emulsion polymerization, suspension polymerization and bulk-suspension polymerization while appropriately selecting a radical polymerization initiator, an anion polymerization catalyst, a coordination anion polymerization catalyst, a cation polymerization catalyst, and the like depending on the type and the like of polymerizable unsaturated monomer and reaction medium used.

In addition, polycondensation of the polycondensable component having an acid-dissociable group can be carried out in a water medium or a mixed medium of water and a hydrophilic solvent, preferably in the presence of an acid catalyst.

In the case where the polymer (B1) is produced by polymerization of a polymerizable unsaturated monomer or through polymerization of a precursor, a branched structure can be introduced by a repeating unit derived from a polyfunctional monomer having two or more polymerizable unsaturated bonds, and/or an acetal type crosslinking group into the polymer (B1). The introduction of such a branched structure enables heat resistance in the polymer (B1) to be enhanced.

In such a case, the introduction rate of a branched structure into the polymer (B1) can be appropriately adopted depending on the type of the branched structure and the polymer introduced. The introduction rate is preferably no greater than 10 mol % with respect to all the repeating units.

Molecular weight of the polymer (B1) is not particularly limited and can be appropriately selected. Polystyrene equivalent weight molecular weight determined by gel permeation chromatography (GPC) (hereinafter, may be also referred to as “Mw”) is typically 1,000 to 500,000, preferably 2,000 to 400,000 and more preferably 3,000 to 300,000.

In addition, the Mw of the polymer (B1) having no branched structure is preferably 1,000 to 150,000 and more preferably 3,000 to 100,000. The Mw of the polymer (B1) having a branched structure is preferably 5,000 to 500,000 and more preferably 8,000 to 300,000. Use of the polymer (B1) having the Mw falling within such a range makes obtained resist excellent in alkali developability.

In addition, the ratio (Mw/Mn) of the Mw of the polymer (B1) to the polystyrene equivalent number average molecular weight determined by GPC (hereinafter, may be also referred to as “Mn”) is also not particularly limited, and is typically 1 to 10, preferably 1 to 8 and more preferably 1 to 5. The Mw/Mn of the polymer (B1) falling within such a range makes a photoresist film excellent in resolving performance.

### 3. (C) Polymer Having a Fluorine Atom:

The radiation-sensitive resin composition of the embodiment of the present invention preferably contains (C) a polymer as a high-molecular additive. In the case where a photoresist film is formed using a radiation-sensitive resin composition containing the polymer (B) and the polymer (C), there is a tendency that the polymer (C) predominantly distributes on a surface of the photoresist film due to oil repellency of the polymer (C). In other words, the polymer (C) unevenly distributes on the surface of the photoresist film. Therefore, there is no necessity to form separately an upper layer film for the purpose of blocking a photoresist film from a liquid immersion medium, so that such a radiation-sensitive resin composition is suitably used in the liquid immersion lithography process.

The polymer (C) is not particularly limited as long as a fluorine atom is included in the polymer, and the polymer (C) preferably has a repeating unit having a fluorine atom (hereinafter, may be also referred to as “repeating unit (C1)”). Specific examples of the repeating unit (C1) include repeating units represented by the following general formulae (C1-1) to (C1-3) (hereinafter, may be also referred to as “repeating unit (C1-1) to (C1-3)”). In the case where the polymer (C) has the repeating unit (C1-1) to (C1-3), elution of the acid generating agent, an acid diffusion control agent, and the like in a photoresist film into a liquid for liquid immersion lithography can be suppressed. In addition, due to an increase of a receding contact angle between the photoresist film and the liquid for liquid immersion lithography, water droplets derived from the liquid for liquid immersion lithography is less likely to remain on the photoresist film, so that generation of defects resulting from the liquid for liquid immersion lithography can be also inhibited.

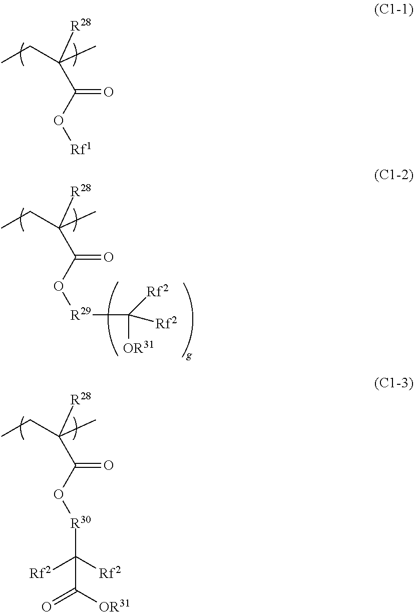

In the general formulae (C1-1) to (C1-3), R²⁸ represents a hydrogen atom, a methyl group, or a trifluoromethyl group. In the general formula (C1-1), Rf¹ represents a hydrocarbon group having 1 to 30 carbon atoms wherein at least one hydrogen atom is substituted with a fluorine atom. In the general formula (C1-2), R²⁹ represents a linking group having a valency of (g+1); and g represents an integer of 1 to 3. In the general formula (C1-3), R³⁰ represents a bivalent linking group. In the general formulae (C1-2) and (C1-3), R³¹ represents a hydrogen atom, a monovalent hydrocarbon group having 1 to 30 carbon atoms, an acid-dissociable group, or an alkali-dissociable group; Rf²s each independently represent a hydrogen atom, a fluorine atom, or a hydrocarbon group having 1 to 30 carbon atoms wherein at least one hydrogen atom is substituted with a fluorine atom, wherein there is no case where all Rf²s are hydrogen atoms.

### (Repeating Unit (C1-1))

In the general formula (C1-1), examples of Rf¹ include linear or branched aliphatic hydrocarbon groups having 1 to 6 carbon atoms wherein at least one hydrogen atom is substituted with a fluorine atom, alicyclic hydrocarbon groups having 4 to 20 carbon atoms wherein at least one hydrogen atom is substituted with a fluorine atom and groups derived therefrom.

Examples of the linear or branched alkyl group having 1 to 6 carbon atoms wherein at least one hydrogen atom is substituted with a fluorine atom include the groups recited as the specific examples of the linear or branched alkyl group having 1 to 7 carbon atoms among the groups represented by R⁴¹ in the above general formula (R⁴-1).

In addition, examples of the alicyclic hydrocarbon group having 4 to 20 carbon atoms wherein at least one hydrogen atom is substituted with a fluorine atom or a group derived therefrom include partially fluorinated or perfluoroalkylated groups of an alicyclic hydrocarbon group such as a cyclopentyl group, a cyclopentylmethyl group, a 1-(1-cyclopentylethyl) group, a 1-(2-cyclopentylethyl) group, a cyclohexyl group, a cyclohexylmethyl group, a 1-(1-cyclohexylethyl) group, a 1-(2-cyclohexylethyl) group, a cycloheptyl group, a cycloheptylmethyl group, a 1-(1-cycloheptylethyl) group, a 1-(2-cycloheptylethyl) group, a 2-norbornyl group, or the like.

Suitable examples of the monomer that provides the repeating unit (C1-1) include trifluoromethyl (meth)acrylic acid esters, 2,2,2-trifluoroethyl (meth)acrylic acid esters, perfluoroethyl (meth)acrylic acid esters, perfluoro n-propyl (meth)acrylic acid esters, perfluoro i-propyl (meth)acrylic acid esters, perfluoro n-butyl (meth)acrylic acid esters, perfluoro i-butyl (meth)acrylic acid esters, perfluoro t-butyl (meth)acrylic acid esters, 2-(1,1,1,3,3,3-hexafluoropropyl) (meth)acrylic acid esters, 1-(2,2,3,3,4,4,5,5-octafluoropentyl)(meth)acrylic acid esters, perfluorocyclohexylmethyl (meth)acrylic acid esters, 1-(2,2,3,3,3-pentafluoropropyl)(meth)acrylic acid esters, 1-(3,3,4,4,5,5,6,6,7,7,8,8,9,9,10,10,10-heptadecafluorodecyl) (meth)acrylic acid esters, 1-(5-trifluoromethyl-3,3,4,4,5,6,6,6-octafluorohexyl)(meth)acrylic acid esters, and the like.

### (Repeating Units (C1-2) and (C1-3))

In the general formulae (C1-2) and (C1-3), R³¹ represents a hydrogen atom or a monovalent organic group. Examples of the monovalent organic group include monovalent hydrocarbon groups having 1 to 30 carbon atoms, acid-dissociable groups, alkali-dissociable groups, and the like.

Examples of the monovalent hydrocarbon group having 1 to 30 carbon atoms include linear or branched aliphatic chain hydrocarbon groups having 1 to 10 carbon atoms and alicyclic hydrocarbon groups having 3 to 30 carbon atoms. These hydrocarbon groups are similar to the linear or branched alkyl groups having 1 to 7 carbon atoms and the alicyclic hydrocarbon group having 3 to 7 carbon atoms described in connection with R⁴¹. In addition, the hydrocarbon groups may have a substituent. As such a substituent, the explanation of the substituent which R¹ to R³ in the above general formula (1) may have can be adopted as is.

In the general formulae (C1-2) and (C1-3), among the groups represented by R³¹, an acid-dissociable group refers to a group which substitutes for a hydrogen atom in a polar functional group such as, for example, a hydroxyl group or a carboxyl group, and which dissociates in the presence of an acid. Specifically, examples of the acid-dissociable group include a t-butoxycarbonyl group, a tetrahydropyranyl group, a tetrahydrofuranyl group, a (thiotetrahydropyranyl sulfanil)methyl group, a (thiotetrahydrofuranyl sulfanil)methyl group, an alkoxy substituted methyl group, an alkylsulfanil substituted methyl group, and the like. It is to be noted that examples of the alkoxyl group (substituent) in the alkoxy substituted methyl group and the alkyl group (substituent) in the alkylsulfanil substituted methyl group include alkoxyl groups having 1 to 4 carbon atoms and alkyl groups having 1 to 4 carbon atoms.

In addition, specific examples of the acid-dissociable group also include groups represented by the following general formula (12).

—C(R³²)₃ (12)

in the general formula (12), three R³²s are identical to R¹⁸ in the general formula (11).

Among these acid-dissociable groups, a group represented by the general formula (12), a t-butoxycarbonyl group, an alkoxy substituted methyl group, and the like are preferable. In the repeating unit (C1-2), a t-butoxycarbonyl group and an alkoxy substituted methyl group are further preferable. In the repeating unit (C1-3), an alkoxy substituted methyl group and the group represented by the general formula (12) are further preferable.

The case where the polymer (C) is the polymer having the repeating unit (C1-2) or (C1-3) having an acid-dissociable group is preferable in terms of a possibility of improving solubility of the polymer (C) at a light-exposed site in a photoresist film. This benefit is considered to result from a reaction of the polymer (C) with an acid generated at the light-exposed site of the photoresist film to generate a polar group in an exposing step in the method for forming a resist pattern described later.

In the general formulae (C1-2) and (C1-3), among the groups represented by R³¹, the alkali-dissociable group refers to a group which substitutes for a hydrogen atom in a polar functional group such as, for example, a hydroxyl group and a carboxyl group, and which dissociates in the presence of an alkali. The alkali-dissociable group is not particularly limited as long as the alkali-dissociable group exhibits the above-mentioned properties, but the group represented by the above general formula (R⁴-1) is preferable in the general formula (C1-2). In addition, groups represented by the above general formulae (R⁴-2) to (R⁴-4) are preferable in the general formula (C1-3).

The case where the polymer (C) is the polymer having the repeating unit (C1-2) or (C1-3) having an alkali-dissociable group is preferable in terms of a possibility of improving the affinity of the polymer (C) to an alkaline developer. This benefit is considered to result from a reaction of the polymer (C) with a developer solution to generate a polar group in a development step in the method for forming a resist pattern.

In the case where the group represented by R³¹ in the general formulae (C1-2) and (C1-3) is a hydrogen atom, repeating units (C1-2) and (C1-3) have a hydroxyl group and a carboxyl group, respectively, which are polar groups. Affinity of the polymer (C) to an alkaline developer can be enhanced in the development step in the method for forming a resist pattern described later when the polymer (C) has such a repeating unit.

In the general formula (C1-2), R²⁹ represents a linking group having a valency of (g+1). Examples of such a linking group include a single bond or hydrocarbon groups having a valency of (g+1) and 1 to 30 carbon atoms. In addition, examples of such a linking group include combinations of any of these hydrocarbon groups with an oxygen atom, a sulfur atom, an imino group, a carbonyl group, a —CO—O— group, or a —CO—NH-group. It is to be noted that g represents an integer of 1 to 3. Provided that g is 2 or 3, the structures represented by the following general formula (C1-2-a) in the general formula (C1-2) are each independent.

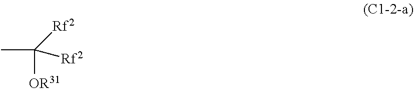

In the general formula (C1-2-a), R³¹ and Rf² are identical to R³¹ and Rf² in the general formula (C1-2).

Examples of R²⁹ having a chain structure include aliphatic hydrocarbon groups having a valency of (g+1) and a structure derived by removing (g+1) hydrogen atoms from an aliphatic hydrocarbon having 1 to 10 carbon atoms such as methane, ethane, propane, butane, 2-methylpropane, pentane, 2-methylbutane, 2,2-dimethylpropane, hexane, heptane, octane, nonane or decane, and the like.

In addition, examples of R²⁹ having a cyclic structure include alicyclic hydrocarbon groups having a valency of (g+1) and a structure derived by removing (g+1) hydrogen atoms from an alicyclic hydrocarbon having 4 to 20 carbon atoms such as cyclobutane, cyclopentane, cyclohexane, bicyclo[2.2.1]heptane, bicyclo[2.2.2]octane, tricyclo[5.2.1.0^{2,6}]decane or tricyclo[3.3.1.1^{3,7}]decane; aromatic hydrocarbon groups having a valency of (g+1) and a structure derived by removing (g+1) hydrogen atoms from an aromatic hydrocarbon having 6 to 30 carbon atoms such as benzene or naphthalene.

Further, among R²⁹s, examples of the structure having an oxygen atom, a sulfur atom, an imino group, a carbonyl group, a —CO—O— group, or a —CO—NH— group include structures represented by the following general formulae (R²⁹-1) to (R²⁹-8):

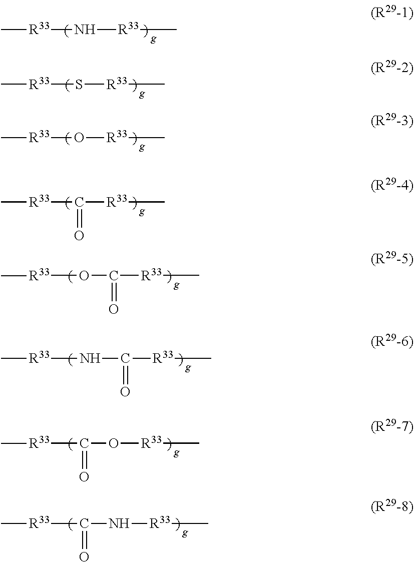

In the general formulae (R²⁹-1) to (R²⁹-8), R³³s each independently represent a single bond, an aliphatic chain hydrocarbon group having 1 to 10 carbon atoms, an alicyclic hydrocarbon group having 4 to 20 carbon atoms, or an aromatic hydrocarbon group having 6 to 30 carbon atoms.

In the general formulae (R²⁹-1) to (R²⁹-8), among the groups represented by R³³, for the aliphatic chain hydrocarbon group having 1 to 10 carbon atoms, the alicyclic hydrocarbon group having 4 to 20 carbon atoms and the aromatic hydrocarbon group having 6 to 30 carbon atoms, the explanation of R²⁹ in the general formula (C1-2-a) can be adopted as is.

In addition, R²⁹s may have substituent(s). For such a substituent, the explanation of the substituents R¹ to R³ in the above general formula (1) may have can be adopted.

In the general formula (C1-3), for the linking group represented by R³⁰, the explanation in the case where g is 1 in the explanation of R²⁹ in the general formula (C1-2-a) can be adopted.

In the general formula (C1-2) or the general formula (C1-3), the hydrocarbon group having 1 to 30 carbon atoms represented by Rf² wherein at least one hydrogen atom is substituted with a fluorine atom is as defined for Rf¹ in the general formula (C1-1).

In the general formulae (C1-2) and (C1-3), examples of the partial structure represented by the following general formula (C1-2-b) include partial structures represented by the following formulae (C1-2-b1) to (C1-2-b5). Among them, in the general formula (C1-2), the partial structure represented by the following formula (C1-2-b5) is preferable, and in the general formula (C1-3), the partial structure represented by the following formula (C1-2-b3) is preferable.

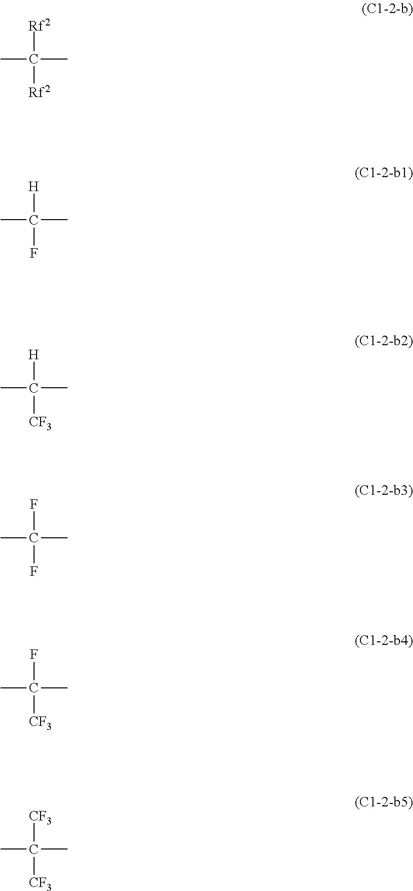

Specific examples of the repeating unit (C1-2) include repeating units represented by the following general formulae (C1-2-1) and (C1-2-2).

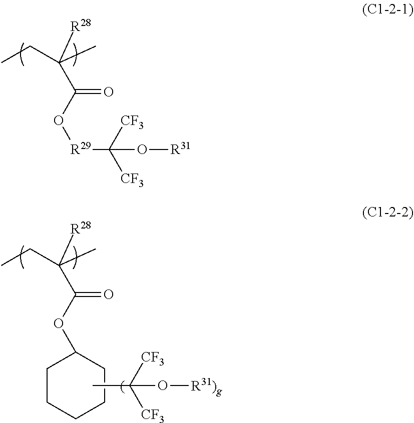

In the general formulae (C1-2-1) and (C1-2-2), R²⁸, R²⁹, R³¹, and g are as defined for R²⁸, R²⁹, R³¹, and g in the general formula (C1-2). Examples of the compound that provides such repeating units include compounds represented by the following general formulae (C1-2-m1) to (C1-2-m5).

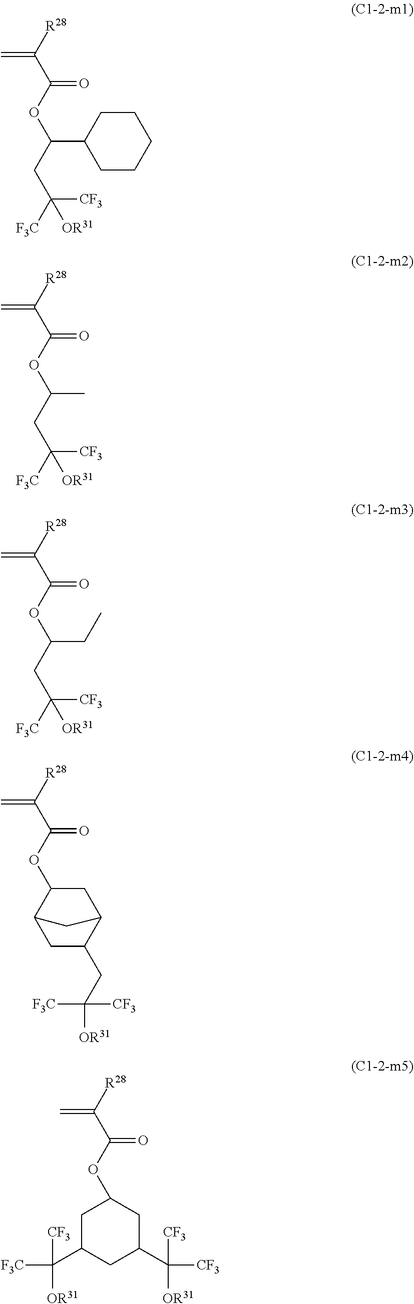

In the general formulae (C1-2-m1) to (C1-2-m5), R²⁸ and R³¹ are as defined in connection with the general formula (C2-1).

With respect to a series of compounds derived from the general formula (C1-2), in the case where a group represented by R³¹ is an acid-dissociable group or an alkali-dissociable group, the compound can be synthesized, for example, using a compound in which R³¹ is a hydrogen atom as a basic ingredient. By way of an example, a compound wherein R³¹ is a group represented by the general formula (R⁴-1) can be formed by fluoroacylating a compound wherein R³¹ is a hydrogen atom with a conventionally well-known method. More specifically, the method may include (1) esterification by condensation of an alcohol and a fluorocarboxylic acid in the presence of an acid, (2) esterification by condensation of an alcohol and a fluorocarboxylic acid halide in the presence of a base, and the like.

Specific examples of the repeating unit (C1-3) include repeating units represented by the following general formula (C1-3-1).

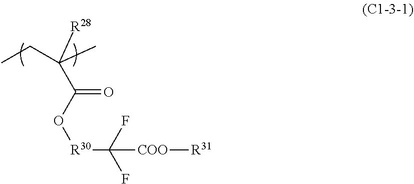

In the general formula (C1-3-1), R²⁸, R³⁰ and R³¹ are as defined in connection with R²⁸, R³⁰ and R³¹ in the general formula (C1-3). Examples of the compound that provides these repeating units include compounds represented by the following general formulae (C1-3-m1) to (C1-3-m4).

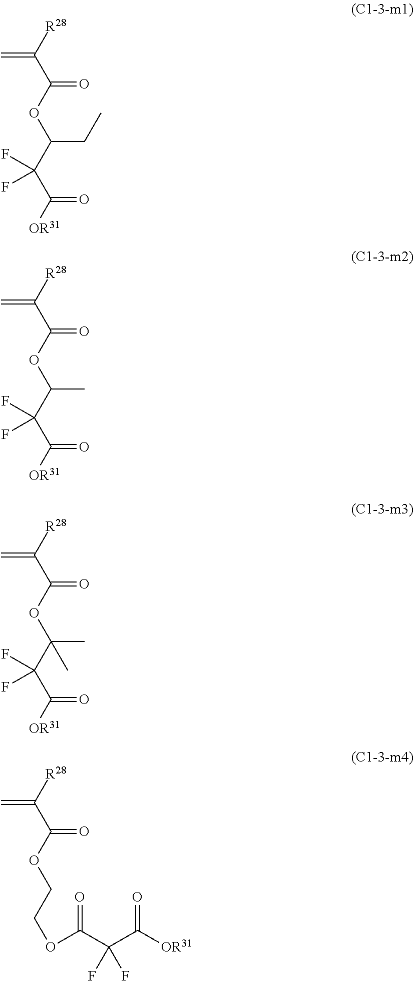

In the general formulae (C1-3-m1) to (C1-3-m4), R²⁸ and R³¹ are as defined in connection with the description of R²⁸ and R³¹ in the general formula (C1-3).

With respect to a series of compounds derived from the general formula (C1-3), in the case where the group represented by R³¹ is an acid-dissociable group or an alkali-dissociable group, the compound can be synthesized, for example, using a compound wherein R³¹ is a hydrogen atom or a derivative thereof as a basic ingredient. By way of an example, a compound wherein R³¹ is represented by the general formula (R⁴-4) can be synthesized by reacting, for example, a compound represented by the following general formula (m-1) with a compound represented by the following general formula (m-2).

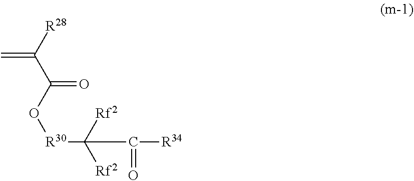

In the general formula (m-1), R²⁸, R³⁰ and Rf² are as defined for R²⁸, R³⁰ and Rf² the general formula (C1-3); and R³⁴ represents a hydroxyl group or a halogen atom.

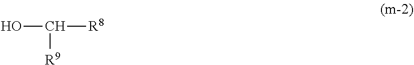

In the general formula (m-2), R⁸ and R⁹ are as defined for R⁸ and R⁹ in the general formula (R⁴-4).

The polymer (C) may have only one type or two types or more of the repeating units (C1-1) to (C1-3) and preferably has two or more types of the repeating units (C1-1) to (C1-3), and particularly preferably have a combination of the repeating unit (C1-2) with the repeating unit (C1-3).

The polymer (C) preferably further has a repeating unit having an acid-dissociable group other than the repeating unit (C1) (hereinafter, may be also referred to as “repeating unit (C2)”), a repeating unit having an alkali-soluble group, excluding those falling under the repeating unit (C1), (hereinafter, may be also referred to as “repeating unit (C3)”), or a repeating unit having a lactone skeleton (hereinafter, may be also referred to as “repeating unit (C4)”).

In the case where a polymer having the repeating unit (C2) is used as the polymer (C), the difference between an advancing contact angle and a receding contact angle of a photoresist film can be small, thereby being capable of coping with an increase in scanning speed upon exposure. Suitable examples of the repeating unit (C2) include the aforementioned repeating unit (11).

Furthermore, the repeating unit (C2) is particularly preferable a repeating unit represented by the general formula (C2-1) among the repeating units (11).

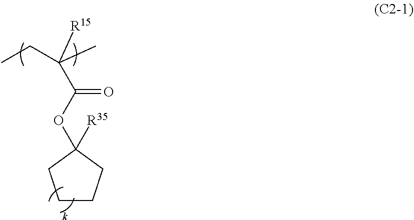

In the general formula (C2-1), R¹⁵ represents a hydrogen atom, a methyl group, or a trifluoromethyl group; R³⁵ represents a linear or branched alkyl group having 1 to 4 carbon atoms; and k represents an integer of 1 to 4.

In the general formula (C2-1), examples of the linear or branched alkyl group having 1 to 4 carbon atoms represented by R³⁵ include a methyl group, an ethyl group, an n-propyl group, an i-propyl group, an n-butyl group, a 2-methylpropyl group, a 1-methylpropyl group, a t-butyl group, and the like.

The polymer (C) may have either one type alone or in combination of two types or more of the repeating unit (C2). Furthermore, in the case where the polymer (C) has the repeating unit (C3) or the repeating unit (C4), solubility in an alkaline developer can be enhanced.

An alkali-soluble group in the repeating unit (C3) is preferably a functional group having a hydrogen atom, and having a pKa of 4 to 11 in view of enhancement in solubility in an alkaline developer. Specific examples of the functional group include functional groups represented by the general formula (C-3a) or the formula (C-3b), and the like.

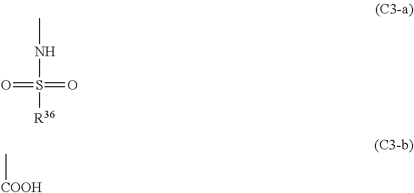

In the general formula (C-3a), R³⁶ represents a hydrocarbon group having 1 to 10 carbon atoms wherein at least one hydrogen atom is substituted with a fluorine atom.

The hydrocarbon group having 1 to 10 carbon atoms wherein at least one hydrogen atom is substituted with a fluorine atom represented by R³⁶ in the general formula (C-3a) is not particularly limited, and a trifluoromethyl group and the like are preferable.

It is to be noted that a main chain skeleton of the repeating unit (C3) is not particularly limited, and is preferably a skeleton of a methacrylic acid ester, an acrylic acid ester, an α-trifluoroacrylic acid ester, or the like.

Examples of the repeating unit (C3) include repeating units derived from the compound represented by the general formulae (C3-a-1) or (C3-b-1).

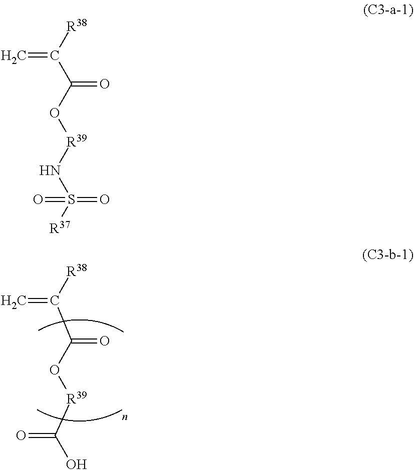

In the general formulae (C3-a-1) and (C3-b-1), R³⁸ represents a hydrogen atom, a methyl group, or a trifluoromethyl group; and R³⁹ represents a single bond or a bivalent saturated or unsaturated hydrocarbon group having 1 to 20 carbon atoms. In the general formula (C3-a-1), R³⁷ represents a hydrocarbon group having 1 to 10 carbon atoms wherein at least one hydrogen atom is substituted with a fluorine atom; and n represents 0 or 1.

The group represented by R³⁹ in the general formulae (C3-a-1) and (C3-b-1) is as defined in connection for R³⁰ in the general formula (C1-3). In addition, the group represented by R³⁷ in the general formula (C3-a-1) is as defined for the general formula (C3-a).

The polymer (C) may have either one type alone or in combination of two or more types of the repeating unit (C3).

An example of the repeating unit (C4) includes a repeating unit (10).

Here, preferable proportions of each repeating unit contained with respect to 100 mol % of the total of all repeating units in the repeating unit (C1) in the polymer (C) are shown below. The proportion of the repeating unit (C1) contained is preferably 20 to 90 mol % and particularly preferably 20 to 80 mol %. The proportion of the repeating unit (C2) contained is typically no greater than 80 mol %, preferably 20 to 80 mol % and more preferably 30 to 70 mol %. The proportion of the repeating unit (C2) contained falling within the range is particularly advantageous in the viewpoint that a difference between an advancing contact angle and a receding contact angle should be made smaller. Further, the proportion of the repeating unit (C3) contained is typically no greater than 50 mol %, preferably 5 to 30 mol % and more preferably 5 to 20 mol %. The proportion of the repeating unit (C4) contained is typically no greater than 50 mol %, preferably 5 to 30 mol % and more preferably 5 to 20 mol %.

The polymer (C) can be prepared, for example, by polymerizing a polymerizable unsaturated monomer corresponding to each predetermined repeating unit in a proper solvent using a radical polymerization initiator such as hydroperoxides, dialkylperoxides, diacylperoxides and azo compounds, in the presence of a chain transfer agent as needed.

Examples of the solvent used in polymerization include alkanes such as n-pentane, n-hexane, n-heptane, n-octane, n-nonane and n-decane; cycloalkanes such as cyclohexane, cycloheptane, cyclooctane, decalin and norbornane; aromatic hydrocarbons such as benzene, toluene, xylene, ethyl benzene and cumene; halogenated hydrocarbons such as chlorobutanes, bromohexanes, dichloroethanes, hexamethylenedibromide and chlorobenzene; saturated carboxylic acid esters such as ethyl acetate, n-butyl acetate, i-butyl acetate and methyl propionate; ketones such as acetone, 2-butanone, 4-methyl-2-pentanone and 2-heptanone; ethers such as tetrahydrofuran, dimethoxy ethanes and diethoxyethanes; alcohols such as methanol, ethanol, 1-propanol, 2-propanol and 4-methyl-2-pentanol, and the like. These solvents can be used either one type alone or as a mixture of two types or more thereof. In addition, the reaction temperature in polymerization is typically 40 to 150° C. and preferably 50 to 120° C. The reaction time is typically 1 to 48 hrs and preferably 1 to 24 hrs.

The Mw of the polymer (C) is preferably 1,000 to 50,000, more preferably 1,000 to 40,000 and further preferably 1,000 to 30,000. When the Mw is less than 1,000, a photoresist film having a sufficient receding contact angle may not be formed. On the other hand, when the Mw is greater than 50,000, developability of a photoresist film may decrease. In addition, the ratio (Mw/Mn) of the Mw to the Mn of the polymer (C) is preferably 1 to 5 and more preferably 1 to 4.

The less the content of impurities such as halogen and metal is, the more preferable the polymer (C) is. Less content of the impurities enables further enhancement in sensitivity, resolution, process stability, pattern configuration, and the like in a photoresist film.

The amount of the polymer (C) blended is preferably 0.1 to 20 parts by mass, more preferably 1 to 10 parts by mass and particularly preferably 1 to 7.5 parts by mass with respect to 100 parts by mass of the polymer (B). When the amount of the polymer (C) blended is no greater than 0.1 parts by mass, efficacy obtained from containing the polymer (C) may not be sufficient. On the other hand, when the amount of the polymer (C) blended is more than 20 parts by mass, poor development may occur since water repellency on a resist surface becomes too high.

The proportion of a fluorine atom in the polymer (C) contained is preferably greater than the proportion of a fluorine atom in the polymer (B). Specifically, the proportion of a fluorine atom in the polymer (C) contained is typically no less than 5% by mass, preferably 5 to 50% by mass and more preferably 5 to 45% by mass with respect to 100% by mass of the total of the polymer (C). It is to be noted that the proportion of a fluorine atom in the polymer (C) contained can be determined by ¹³C-NMR. In the case where the proportion of a fluorine atom in the polymer (C) contained is greater than the proportion of a fluorine atom in the polymer (B), water repellency on the surface of a photoresist film formed by a radiation-sensitive resin composition containing the polymer (C) and the polymer (B) can be enhanced, thereby eliminating a necessity of separately forming an upper layer film in liquid immersion lithography. It is to be noted that a difference between the proportion of a fluorine atom in the polymer (C) contained and the proportion of a fluorine atom in the polymer (B) is preferably no less than 1% by mass and more preferably no less than 5% by mass in order to achieve the above-mentioned effects sufficiently.

### 4. Additive:

With the radiation-sensitive resin composition of the embodiment of the present invention, conventionally well-known additives may be blended as needed. Preferable additives include acid diffusion control agents having a controlling action of a diffusion phenomenon in a photoresist film, of an acid generated from an acid generating agent by exposure and a suppressing action of an undesirable chemical reaction in an unexposed region. Blending the acid diffusion control agent enables storage stability of the radiation-sensitive resin composition to be enhanced, and the resolution to be further enhanced. In addition, line width variation of a resist pattern by fluctuation of post exposure time delay (PED) from exposure to development treatment can be also prevented. Therefore, a radiation-sensitive resin composition which is extremely superior in process stability can be obtained.

Specific example of the acid diffusion control agent includes the nitrogen-containing organic compound described in paragraph nos. 0176 to 0187 of PCT International Publication No. 2009/051088.

Examples of the nitrogen-containing organic compound include trialkylamines such as tri-n-hexylamine, tri-n-heptylamine and tri-n-octylamine; nitrogen-containing organic compounds having an acid-dissociable group such as N-t-butoxycarbonyl-4-hydroxypiperidine, N-t-butoxycarbonylpyrrolidine and N-t-butoxycarbonyl-N′,N″dicyclohexylamine; a polyethyleneimine, polyallylamine, polymers of dimethylaminoethyl acrylamide; nitrogen-containing heterocyclic compound such as 2-phenylbenzimidazole and N-t-butoxycarbonyl-2-phenylbenzimidazole; N,N,N′,N′-tetrakis(2-hydroxypropyl)ethylenediamine, and the like. It is to be noted that the nitrogen-containing organic compounds may be used either alone or as a mixture of two types or more thereof.

In addition, as the acid diffusion control agent, a compound represented by the general formula (D1-0) may be also used.

X⁺Z⁻ (D1-0)

In the general formula (D1-0), X⁺ represents a cation represented by the general formula (D1-1), or a cation is represented by the general formula (D1-2); Z⁻ represents OH⁻, an anion represented by, R^{D1}—COO⁻, an anion represented by R^{D1}—SO₃⁻, or an anion represented by R^{D1}—N⁻—SO₂—R^{D21}, wherein R^{D1} represents an alkyl group which may be substituted, a monovalent alicyclic hydrocarbon group, or an aryl group, and R^{D21} represents a fluorinated aliphatic chain hydrocarbon group which may be substituted, or a monovalent fluorinated alicyclic hydrocarbon group.

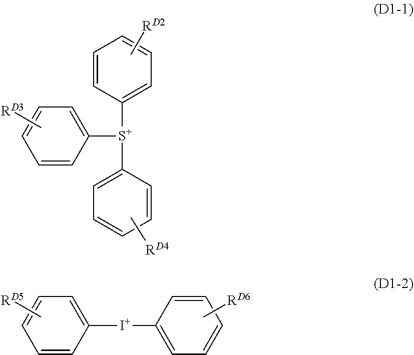

In the general formula (D1-1), R^{D2} to R^{D4} each independently represent a hydrogen atom, an alkyl group, an alkoxyl group, a hydroxyl group, or a halogen atom. In the general formula (D1-2), R^{D5} and R^{D6} each independently represent a hydrogen atom, an alkyl group, an alkoxyl group, a hydroxyl group, or a halogen atom.

The compound represented by the general formula (D1-0) is used as an acid diffusion control agent which is degraded by exposure to lose acid diffusion controllability (hereinafter, may be also referred to as “photodegradable acid diffusion control agent”). Inclusion of the compound makes an acid diffused at a light-exposed site and controls diffusion of an acid at a light-exposed site, thereby resulting in an excellent contrast between the light-exposed site and the light-unexposed site (i.e., making a boundary part between the light-exposed site and the light-unexposed site clear). Therefore, particularly LWR and MEEF of the radiation-sensitive resin composition of the embodiment of the present invention can be effectively improved.

R^{D2} to R^{D4} in the general formula (D1-1) each independently represent a hydrogen atom, an alkyl group, an alkoxyl group, a hydroxyl group, or a halogen atom. Among them, from a viewpoint of decreasing solubility in a developer solution, R^{D2} to R^{D4} in the general formula (D1-1) are preferably a hydrogen atom, an alkyl group, an alkoxy group, or a halogen atom. In addition, R^{D5} and R^{D6} in the general formula (D1-2) each independently represent a hydrogen atom, an alkyl group, an alkoxyl group, a hydroxyl group, or a halogen atom. Among them, R^{D5} and R^{D6} in the general formula (D1-2) are preferably a hydrogen atom, an alkyl group, or a halogen atom.

Z⁻ in the general formula (D1-0) is OH⁻, an anion represented by R^{D1}—COO⁻, R^{D1}—SO₃⁻, or an anion represented by R^{D1}—N⁻−SO₂—R^{D21}, wherein R^{D1} represents an alkyl group which may be substituted, a monovalent alicyclic hydrocarbon group, or an aryl group, and R^{D21} represents a fluorinated aliphatic chain hydrocarbon group which may be substituted, or a monovalent fluorinated alicyclic hydrocarbon group.

It is to be noted that Z⁻ in the general formula (D1-0) is preferably an anion represented by the following formula (D1-3) (i.e., an anion wherein R^{D1} is a phenol group), or an anion represented by the following formula (D1-4) (i.e., an anion wherein R^{D1} is a group derived from 1,7,7-trimethyl bicyclo[2.2.1]heptan-2-one).

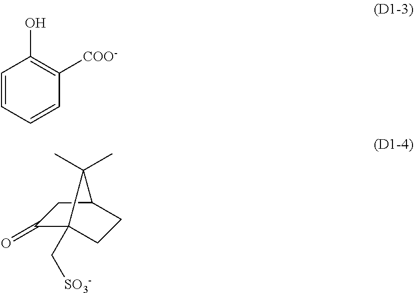

The photodegradable acid diffusion control agent is represented by the general formula (D1-0), and specifically is a sulfonium salt compound or an iodonium salt compound which meets the above-mentioned requirements.

Specific examples of the sulfonium salt compound include triphenylsulfonium hydroxide, triphenylsulfonium acetate, triphenylsulfonium salicylate, diphenyl-4-hydroxyphenylsulfonium hydroxide, diphenyl-4-hydroxyphenylsulfonium acetate, diphenyl-4-hydroxyphenylsulfonium salicylate, triphenylsulfonium 10-camphorsulfonate, 4-t-butoxyphenyl.diphenyl sulfonium 10-camphorsulfonate, and the like. It is to be noted that these sulfonium salt compounds may be used either alone or as a mixture of two or more thereof.

In addition, specific examples of the iodonium salt compound include bis(4-t-butylphenyl)iodonium hydroxide, bis(4-t-butylphenyl)iodonium acetate, bis(4-t-butylphenyl)iodonium hydroxide, bis(4-t-butylphenyl)iodonium acetate, bis(4-t-butylphenyl)iodonium salicylate, 4-t-butylphenyl-4-hydroxyphenyliodonium hydroxide, 4-t-butylphenyl-4-hydroxyphenyliodonium acetate, 4-t-butylphenyl-4-hydroxyphenyliodonium salicylate, bis(4-t-butylphenyl)iodonium 10-camphorsulfonate, diphenyl iodonium 10-camphorsulfonate, and the like. It is to be noted that these iodonium salt compounds may be used either alone or as a mixture of two or more thereof.

The amount of the acid diffusion control agent blended is preferably no greater than 15 parts by mass, more preferably 0.001 to 10 parts by mass and particularly preferably 0.005 to 5 parts by mass with respect to 100 parts by mass of the polymer (B). The amount of the acid diffusion control agent blended of no less than 0.001 parts by mass enables impairment of a pattern configuration and a decrease in dimension fidelity depending on process conditions to be prevented. In addition, the amount of the acid diffusion control agent blended of no greater than 15 parts by mass enables further enhancement in sensitivity as a resist and alkali developability.

Also, a dissolution control agent having a property of enhancing the solubility in an alkaline developer by an action of an acid may be blended. The dissolution control agent include is exemplified by a compound having an acidic functional group such as a phenolic hydroxyl group, a carboxyl group or a sulfonic acid group, a compound derived by substituting a hydrogen atom of an acidic functional group in the compound with an acid-dissociable group, and the like.

The dissolution control agent may be a low-molecular compound, or a high-molecular compound. In the case where the radiation-sensitive resin composition is a negative type radiation-sensitive resin composition, the polymer (B1) may be used as the high-molecular dissolution control agent. It is to be noted that the dissolution control agent may be used either alone or as a mixture two or more thereof. The amount of the dissolution control agent blended is typically no greater than 50 parts by mass and preferably no greater than 20 parts by mass with respect to 100 parts by mass of the polymer (B).

Further, a surfactant that exhibits an action to improve a coating property, striation, developability, and the like may be also blended. As the surfactant, any of an anion-based surfactant, a cation surfactant, a nonionic surfactant and an amphoteric ion surfactant may be used, and the surfactant is preferably a nonionic surfactant. It is to be noted that the surfactant may be used either alone or as a mixture of two or more thereof. The amount of the surfactant blended, in terms of an active ingredient of the surfactant, is typically no greater than 2 parts by mass and preferably no greater than 1.5 parts by mass with respect to 100 parts by mass of the polymer (B).

Examples of the nonionic surfactant include polyoxyethylene higher alkyl ethers, polyoxyethylene higher alkyl phenyl ethers and higher aliphatic acid diesters of polyethylene glycol, as well as each series of the following trade names, “KP” (manufactured by Shin-Etsu Chemical Co., Ltd.), “Polyflow” (manufactured by Kyoeisha Chemical Co., Ltd.), “EFTOP” (manufactured by Mitsubishi Materials Electronic Chemicals Co., Ltd.), “MEGAFACE” (manufactured by Dainippon Ink and Chemicals, Incorporated), “Fluorad” (manufactured by Sumitomo 3M Limited), “AsahiGuard” and “Surflon” (manufactured by Asahi Glass Co., Ltd.), and the like.

In addition, a sensitizer can be also blended which absorbs energy of a radioactive ray and transmits the energy to the acid generating agent, thereby achieving an increasing action of the amount of the acid generated and enabling the apparent sensitivity to be enhanced. Examples of the sensitizer include acetophenones, benzophenone, naphthalenes, biacetyl, eosine, rose bengal, pyrenes, anthracenes, phenothiazines, and the like. These sensitizers may be used either alone or as a mixture of two or more thereof. The amount of the sensitizer blended is typically no greater than 50 parts by mass and preferably no greater than 30 parts by mass with respect to 100 parts by mass of the polymer (B).

Further, (G) a lactone compound may be also blended which has an effect to efficiently segregate on the surface of a resist film the polymer (C) that exhibits an action to permit expression of water repellency on the surface of the resist film in liquid immersion lithography. Blending the lactone compound (G) enables the amount of the added polymer (C) to be decreased when the polymer (C) is included. Therefore, elution of a component from a photoresist film to a liquid for immersion lithography liquid can be inhibited without deteriorating basic characteristics of the resist, and no droplets remain even if liquid immersion lithography is carried out by high speed scanning. As a result, water repellency of the surface of the resist film which suppresses defects resulting from liquid immersion such as watermark defects can be maintained.

Specific examples of the lactone compound (G) include γ-butyrolactone, valerolactone, mevalonic lactone, norbornane lactone, and the like. It is to be noted that the lactone compound (G) may be blended either alone or two or more thereof. The amount of the lactone compound (G) blended is typically 30 to 200 parts by mass and more preferably 50 to 150 parts by mass with respect to 100 parts by mass of the polymer (B). When the amount of the lactone compound (G) blended is too small, addition of a small amount of the polymer (C) leads to failure in sufficiently obtaining water repellency on the resist film surface. On the other hand, when the amount of the polymer (C) blended is too large, basic characteristics of the resist and pattern configuration obtained after development may be significantly deteriorated.

In addition, additives other than the above-mentioned additives such as, for example, a dye, a pigment, an adhesion promoter, a halation inhibitor, a preservation stabilizer, a defoaming agent and a shape improving agent, and specifically 4-hydroxy-4′-methylchalcone and the like can be blended as needed within the range not to inhibit effects of the embodiment of the present invention. In such a case, blending of a dye and/or a pigment enables visualization of a latent image at a light-exposed site and amelioration of an effect of halation upon exposure. In addition, blending an adhesion promoter enables adhesiveness to a substrate to be improved.

### Preparation Method

The radiation-sensitive resin composition of the embodiment of the present invention is prepared as a composition solution typically by dissolving each component into (E) a solvent in use to form a homogenous solution, and thereafter filtering with a filter or the like having a pore size of, for example, about 0.2 μm as needed.

The solvent (E) is exemplified by ethers, esters, ether esters, ketones, ketone esters, amides, amide esters, lactams, (halogenated)hydrocarbons, and the like. More specifically, examples of the solvent (E) include ethylene glycol monoalkyl ethers, diethylene glycol dialkyl ethers, propylene glycol monoalkyl ethers, propylene glycol dialkyl ethers, ethylene glycol monoalkyl ether acetates, propylene glycol monoalkyl ether acetates, acyclic or cyclic ketones, acetic acid esters, hydroxyacetic acid esters, alkoxyacetic acid esters, acetoacetic acid esters, propionic acid esters, lactic acid esters, other substituted propionic acid esters, (substituted)butyric acid esters, pyruvic acid esters, N,N-dialkylformamides, N,N-dialkylacetamides, N-alkylpyrrolidones, (halogenated) aliphatic hydrocarbons, (halogenated) aromatic hydrocarbons, and the like.

Specific examples of the solvent (E) include solvents described in paragraph no. 0202 of PCT International Publication No. 2009/051088.

Among these solvents, propylene glycol monoalkyl ether acetates, acyclic or cyclic ketones, lactic acid esters, 3-alkoxypropionic acid esters, and the like are preferable in that favorable film in-plane uniformity can be secured in application. The solvent (E) may be used either alone or as a mixture of two types or more thereof.

In addition, together with the solvent (E), other solvent, for example, a high-boiling solvent such as benzyl ethyl ether, di-n-hexyl ether, diethylene glycol monomethyl ether, diethylene glycol monoethyl ether, acetonyl acetone, isophorone, caproic acid, caprylic acid, 1-octanol, 1-nonanol, benzyl alcohol, benzyl acetate, ethyl benzoate, diethyl oxalate, diethyl maleate, ethylene carbonate, propylene carbonate, ethylene glycol monophenyl ether acetate, or the like may be used as needed.

The other solvent(s) may be used either alone or as a mixture of two types or more thereof. The proportion of the other solvent used is typically no greater than 50% by mass and preferably no greater than 30% by mass with respect to all the solvents.

The amount of the solvent (E) used is the amount that gives the concentration of the total solid content of a composition solution being typically 5 to 50% by mass, preferably 10 to 50% by mass, more preferably 10 to 40% by mass, further preferably 10 to 30% by mass and particularly preferably 10 to 25% by mass. The concentration of the total is solid content of the composition solution falling within the range enables favorable film in-plane uniformity to be secured in application.

### III. Method for Forming a Resist Pattern:

According to the method for forming a resist pattern of the embodiment of the present invention, first, a photoresist film is formed by applying a composition solution prepared as mentioned above by a proper application means for spin-coating, cast coating, roll coating or the like, for example, on a substrate such as a silicon wafer or a wafer coated with aluminum. Then after optionally a heat treatment (hereinafter, may be also referred to as “PB”) is carried out in advance, the photoresist film is exposed through a predetermined mask pattern.

Examples of the radioactive ray capable of being used in exposure include far ultraviolet rays such as a bright line spectrum of a mercury lamp (wavelength: 254 nm), a KrF excimer laser (wavelength: 248 nm), an ArF excimer laser (wavelength: 193 nm), an F₂ excimer laser (wavelength: 157 nm) and an EUV (wavelength: 13 nm and the like), X-rays such as a synchrotron radioactive ray, charged particle rays such as an electron beam, which may be selected depending on the type of the acid generating agent. Among them, far ultraviolet rays and charged particle rays are preferable, and a KrF excimer laser (wavelength: 248 nm), an ArF excimer laser (wavelength: 193 nm), an F₂ excimer laser (wavelength: 157 nm) and an electron beam are particularly preferable. In addition, it is preferable that a liquid for liquid immersion lithography is placed on a photoresist film, and the photoresist film is subjected to liquid immersion lithography through the liquid for liquid immersion lithography.

In addition, exposure conditions such as a radiation dosage are appropriately predetermined depending on the blend composition of the radiation-sensitive resin composition, the type of additives, and the like. Furthermore, in forming a resist pattern, carrying out post-exposure baking (hereinafter, may be also referred to as “PEB”) is preferable in that apparent sensitivity of a resist is enhanced. Baking conditions in PEB vary depending on the blend composition of the radiation-sensitive resin composition, the types of additives, and typically involve 30 to 200° C. and preferably 50 to 150° C.

Thereafter, thus exposed photoresist film is developed with an alkaline developer to form a predetermined positive type or negative type resist pattern.

Examples of the alkaline developer used include alkaline aqueous solutions in which one type or more of alkaline compounds such as, for example, alkali metal hydroxides, ammonia, alkylamines, alkanolamines, heterocyclic amines, tetraalkylammonium hydroxides, choline, 1,8-diazabicyclo[5.4.0]-7-undecene and 1,5-diazabicyclo[4.3.0]-5-nonene is dissolved. Particularly preferable alkaline developer is an aqueous solution of a tetraalkylammonium hydroxide.

The concentration of the alkaline aqueous solution is preferably no greater than 10% by mass, more preferably 1 to 10% by mass and particularly preferably 2 to 5% by mass. The concentration of the alkaline aqueous solution of no greater than 10% by mass enables dissolution of a light-unexposed site (for positive type) or a light-exposed site (for negative type) in the alkaline developer to be inhibited.

In addition, to a developer solution containing an alkaline aqueous solution, a proper amount of a surfactant and the like is preferably added, thereby enabling wettability of the alkaline developer on a photoresist film to be enhanced. It is to be noted that after development with a developer solution containing an alkaline aqueous solution, the photoresist film is generally washed with water and dried.

### EXAMPLES

Hereinafter, the present invention will be specifically described by way of Examples, but the present invention is not limited to the Examples. It is to be noted that “part” and “%” in the Examples, unless particularly stated otherwise, are based on mass basis.

### Synthesis Example 1

### Synthesis of Precursor

(A) As a precursor of a sulfonyl compound, a compound represented by the following formula (a1), diphenyl(4-(3,3,3-trifluoropropanoyloxy)phenyl)sulfonium chloride was synthesized by a method as shown below.

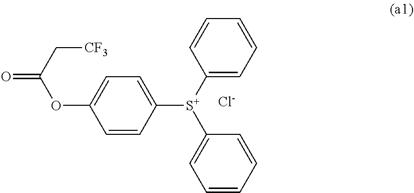 [SMILES: O=C(CC(F)(F)F)Oc1ccc([S+](c2ccccc2)c2ccccc2)cc1 ; [Cl-]]

Into a flask, a mixture of 31.5 g of (4-hydroxyphenyl)diphenylsulfonium chloride, 115 g of trifluoromethyl acetic acid anhydride, and 100 mL of trifluoroacetic acid was added and the mixture was stirred under nitrogen at room temperature for 4 hrs. Trifluoroacetic acid was removed in vacuo, and then 1,000 mL of ethyl acetate was added. Reaction liquid that was washed three times with 1,000 mL of water was concentrated in vacuo to obtain 42.1 g of diphenyl(4-(3,3,3-trifluoropropanoyloxy)phenyl)sulfonium chloride.

The obtained diphenyl(4-(3,3,3-trifluoropropanoyloxy)phenyl)sulfonium chloride was analyzed by NMR (trade name: JNM-EX270, manufactured by JEOL, Ltd.). As a result, the resulting chemical shift was ¹H-NMR δ ppm (CD₃OD): 3.45 (2H), 6.98-7.10 (2H), 7.47-7.89 (12H)), ¹⁹F-NMR (δ ppm (DMSO): 0.53) and was confirmed to be a target compound. It is to be noted that 0 ppm (internal standard) was defined as a peak of sodium 3-trimethylsilylpropionate 2-2,2,3,3-d₄) for ¹H-NMR and benzotrifluoride for ¹⁹F-NMR. Purity was 99% (as determined by ¹H-NMR).

### Example 1

### Synthesis of (A-1) Sulfonium Compound

A compound represented by the following formula (A-1), diphenyl(4-(3,3,3-trifluoropropanoyloxy)phenyl)sulfonium-1,1,2,2,3,3,4,4,4-nonafluorobutane-1-sulfonate, was synthesized by the following method.

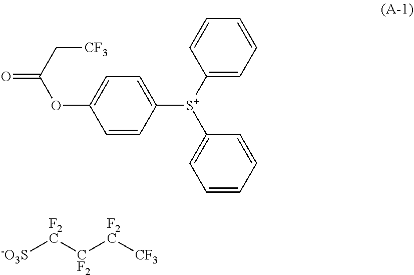

Into a flask, 20.0 g of sodium 1,1,2,2,3,3,4,4,4-nonafluorobutane-1-sulfonate, 28.8 g of diphenyl(4-(3,3,3-trifluoropropanoyloxy)phenyl)sulfonium chloride synthesized in Synthesis Example 1, 100 g of ion exchanged water and 100 g of dichloromethane were charged and the mixture was stirred at room temperature for 1 hour. An organic layer was extracted and washed five times with 100 g of ion exchanged water. Thereafter, the solvent was removed to obtain 34.6 g of diphenyl(4-(3,3,3-trifluoropropanoyloxy)phenyl)sulfonium-1,1,2,2,3,3,4,4,4-nonafluorobutane-1-sulfonate.

The obtained diphenyl(4-(3,3,3-trifluoropropanoyloxy)phenyl)sulfonium-1,1,2,2,3,3,4,4,4-nonafluorobutane-1-sulfonate was analyzed using NMR. As a result, obtained chemical shift was ¹H-NMR δ ppm (DMSO): 3.39 (2H), 6.80-7.21 (2H), 7.22-8.09 (12H)), HRMS Calcd. for C₂₅H₁₈F₁₂O₅S₂: 688.025 (M⁺), Found: 688.025 and was confirmed to be a target compound. It is to be noted that for ¹H-NMR, the peak of sodium 3-trimethylsilylpropionate 2-2,2,3,3-d₄ was defined as 0 ppm (internal standard). Purity was no less than 99% (as determined by ¹H-NMR).

### Example 2

### Synthesis of (A-2) Sulfonium Compound

Using sodium 2-(bicyclo[2.2.1]2-heptanyl-1,1,2,2-tetrafluoroethanesulfonate in place of sodium 1,1,2,2,3,3,4,4,4-nonafluorobutane-1-sulfonate as a starting material, diphenyl(4-(3,3,3-trifluoropropanoyloxy)phenyl)sulfonium-2-(bicyclo[2.2.1]2-heptanyl)-1,1,2,2-tetrafluoroethanesulfonate represented by the following formula (A-2) was synthesized by a method similar to Example 1.

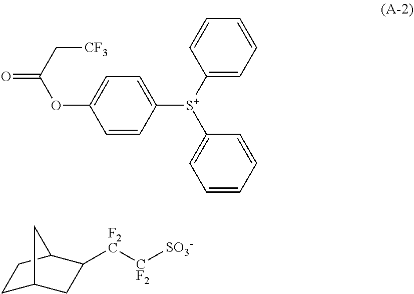

### Example 3

### Synthesis of (A-3) Sulfonium Compound

Using sodium 1,1-difluoro-2-(1-adamantyl)ethane-1-sulfonate in place of sodium 1,1,2,2,3,3,4,4,4-nonafluorobutane-1-sulfonate as a starting material, diphenyl(4-(3,3,3-trifluoropropanoyloxy)phenyl)sulfonium-1,1-difluoro-2-(1-adamantyl)ethane-1-sulfonate represented by the following formula (A-3) was synthesized by a method similar to Example 1.

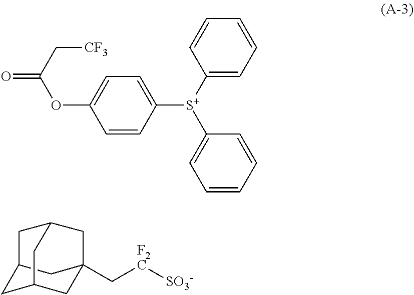

### Example 4

### Synthesis of (A-4) Sulfonium Compound

Using sodium 6-(1-adamantane carbonyloxy)-1,1,2,2-tetrafluorohexane-1-sulfonate in place of sodium 1,1,2,2,3,3,4,4,4-nonafluorobutane-1-sulfonate as a starting material, diphenyl (4-(3,3,3-trifluoropropanoyloxy)phenyl)sulfonium-6-(1-adamantane carbonyloxy)-1,1,2,2-tetrafluorohexane-1-sulfonate represented by the following formula (A-4) was synthesized by a method similar to Example 1.

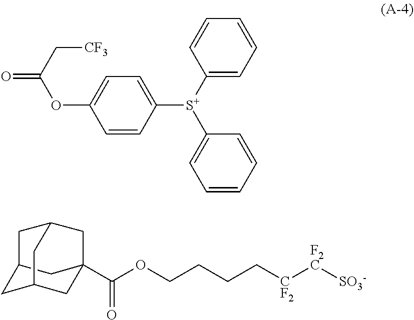

### Example 5

### Synthesis of (A-5) Sulfonium Compound

Using 4-(2,2,3,3,4,4,5,5,6,6,7,7,8,8,9,9,9-heptadecafluorononanoyloxy)phenyl)dimethylsulfonium chloride and sodium trifluoromethanesulfonate in place of diphenyl(4-(3,3,3-trifluoropropanoyloxy)phenyl)sulfonium chloride and sodium 1,1,2,2,3,3,4,4,4-nonafluorobutane-1-sulfonate, respectively, as starting materials, 4-(2,2,3,3,4,4,5,5,6,6,7,7,8,8,9,9,9-heptadecafluorononanoyloxy)phenyl)dimethylsulfonium-trifluoromethanesulfonate represented by the following formula (A-5) was synthesized by a method similar to Example 1.

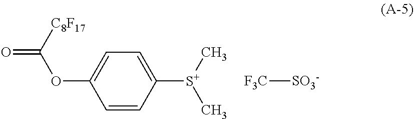

### Preparation of the (B) Polymer

The polymer (B) was prepared using compounds (M-1) to (M-4) represented by the following formulae.

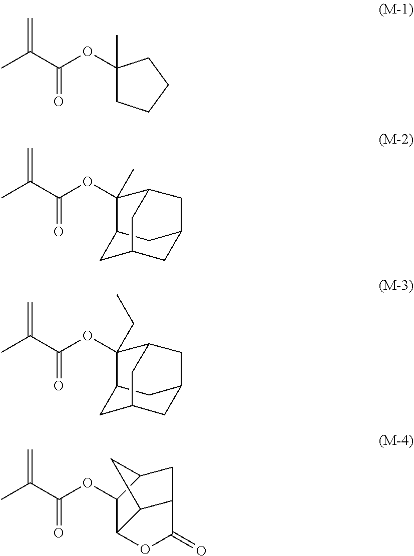

### Synthesis Example 2

### Preparation of (B-1) Polymer

A monomer solution was prepared in which 16.4 g (98 mmol) of the compound (M-1), 5.73 g (24 mmol) of the compound (M-2) and 21.74 g (98 mmol) of the compound (M-4) were dissolved in 100 g of 2-butanone and further 2.01 g of dimethyl 2,2′-azobis(2-methylpropionate) was charged. A 500 mL three-neck flask in which 50 g of 2-butanone and 6.07 g (24 mmol) of compound (M-3) had been charged was purged with nitrogen for 30 min, and then a reaction tank was heated to 80° C. with stirring. A monomer solution prepared in advance was added dropwise for 3 hrs using a dropping funnel. A time point at which the dropwise addition was started was defined as a polymerization starting time, and the polymerization reaction was carried out for 6 hrs.

After completing the polymerization, the polymerization solution was cooled to no higher than 30° C. by water-cooling. The polymerization solution was charged into 1,000 g of methanol and the deposited white powder was filtered out. The filtered white powder was dispersed in 200 g of methanol to give a slurry form, followed by an operation twice including washing and then filtering. The white powder was dried at 50° C. for 17 hrs to obtain a copolymer as a white powder (yield in terms of the weight: 39 g; yield: 78%). The copolymer had the Mw of 6,100, and the Mw/Mn of 1.4, and as a result of ¹³C-NMR analysis, had the contents (mol %) of the repeating units derived from the compound (M-1), the compound (M-2), the compound (M-3) and the compound (M-4) of 42.2, 8.1, 8.4 and 41.3, respectively. The copolymer is designated as (B-1) polymer.

### Preparation of Polymer (C)

The polymer (C) was prepared using compounds (S-1) to (S-9) represented by the following formulae.

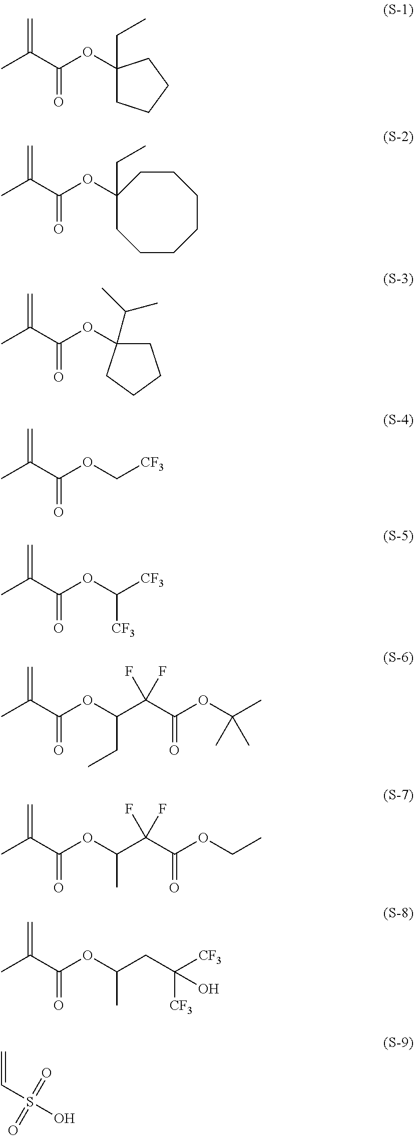

### Synthesis Example 3

### Preparation of (C-1) Polymer

A monomer solution was prepared in which 35.01 g (208 mmol) of the compound (S-1) and 14.99 g (89 mmol) of compound (S-4) were dissolved in 100 g of 2-butanone and further 3.91 g of dimethyl 2,2′-azobis(2-methylpropionate) was charged. On the other hand, 30 g of 2-butanone was charged in a 500 mL three-necked flask, which was purged with nitrogen for 30 min, and then a reaction tank was heated to 80° C. with stirring. Thereafter, using a dropping funnel, the monomer solution prepared in advance was added dropwise for 3 hrs. A time point at which the dropwise addition was started was defined as a polymerization starting time, and the polymerization reaction was carried out for 6 hrs.

After completing the polymerization, the polymerization solution was cooled to no higher than 30° C. by water-cooling, and the polymerization solution was transferred to a 2 L separatory funnel. The polymerization solution was diluted with 150 g of methanol, and 600 g of hexane was charged into the polymerization solution, followed by mixing. Then 21 g of distilled water was charged, and further stirred the reaction mixture and the mixture was allowed to stand for 30 min. Thereafter, an under layer was recovered to give a propylene glycol monomethyl ether acetate solution. The yield of the solid content (polymer) of the propylene glycol monomethyl ether acetate solution was 71%, and the solid content of propylene glycol monomethyl ether acetate solution (polymer) had the Mw of 7,100, the Mw/Mn of 1.3. As a result of ¹³C-NMR analysis, the contents (mol %) of the repeating units derived from the compound (S-1) and the compound (S-4) were 69.8 and 30.2, respectively, and the content of a fluorine atom was 10.2%. The copolymer is defined as (C-1) polymer.

### Synthesis Examples 4 to 5

### Preparation of (C-2) Polymer and (C-3) Polymer

(C-2) a polymer and (C-3) a polymer were prepared in a manner similar to Synthesis Example 3 except that a formulation of compounds was as described in Table 1. Physical properties of the polymer (C-2) and the polymer (C-3) are also listed in Table 1.

TABLE 1

| | | | | | | | (C) Polymer | | | |
|---|---|---|---|---|---|---|---|---|---|---|
| | Monomer 1 | | Monomer 2 | | Monomer 3 | | | | | Content |
| | | Content of | | Content of | | Content of | | | | proportion of |
| | | repeating | | repeating | | repeating | | | | a fluorine |
| | | units | | units | | units | | | | atom |
| | type | (mol %) | type | (mol %) | type | (mol %) | type | Mw | Mw/Mn | (%) |
| Synthesis | S-1 | 69.8 | S-4 | 30.2 | — | — | C-1 | 7,100 | 1.30 | 10.2 |
| Example 3 | | | | | | | | | | |
| Synthesis | S-3 | 61.3 | S-6 | 23.9 | S-8 | 14.8 | C-2 | 6,300 | 1.34 | 9.0 |
| Example 4 | | | | | | | | | | |
| Synthesis | S-2 | 39.5 | S-7 | 60.5 | — | — | C-3 | 6,530 | 1.37 | 9.2 |
| Example 5 | | | | | | | | | | |

### Synthesis Example 6

### Preparation of Polymer for an Upper Layer Film (1)

A monomer solution (i) in which 22.26 g of the compound (S-5) and 4.64 g of 2,2-azobis(methyl 2-methylisopropionate) had been dissolved beforehand in 25 g of methyl ethyl ketone, and a monomer solution (ii) in which 27.74 g of the compound (S-8) had been dissolved beforehand in 25 g of methyl ethyl ketone were prepared, respectively. On the other hand, 100 g of methyl ethyl ketone was charged in a 500 mL three-necked flask equipped with a thermometer and a dropping funnel, and nitrogen was purged for 30 min. After the nitrogen purge, the mixture was heated to 80° C. while stirring in the flask by a magnetic stirrer.

The monomer solution (i) which had been prepared beforehand was added dropwise for 20 min using a dropping funnel, and aged for 20 min. Subsequently, the monomer solution (ii) was added dropwise over 20 min. Thereafter, the reaction was allowed for additional 1 hour and the mixture was cooled to no higher than 30° C. to obtain a copolymerization liquid. The obtained copolymerization liquid was concentrated to 150 g, and then was transferred to a separatory funnel. 50 g of methanol and 400 g of n-hexane were charged in the separatory funnel, and separation and purification were carried out. After the separation, an under layer liquid was recovered. The solvent in the recovered under layer liquid was substituted with 4-methyl-2-pentanol to form a resin solution. The copolymer contained in the obtained resin solution had the Mw of 5,730 and the Mw/Mn of 1.23, and the yield of the copolymer contained in the obtained resin solution was 26%. In addition, the contents (mol %) of the repeating units derived from the compound (S-5) and the compound (S-8) were 50.3 and 49.7, respectively, and the content proportion of a fluorine atom was 43.6%. The copolymer is designated as a polymer for an upper layer film (1).

### Synthesis Example 7

### Preparation of Polymer for an Upper Layer Film (2)

A monomer solution was prepared in which 46.95 g (85 mol %) of the compound (S-8) and 6.91 g of 2,2′-azobis-(methyl 2-methylpropionate) were dissolved in 100 g of isopropyl alcohol. On the other hand, 50 g of isopropyl alcohol was charged in a 500 mL three-necked flask equipped with a thermometer and a dropping funnel, and nitrogen was purged for 30 min. After the nitrogen purge, the mixture was heated to 80° C. while stirring in the flask by a magnetic stirrer. The monomer solution which had been prepared beforehand was added dropwise over 2 hrs using a dropping funnel.

After completing the dropwise addition, the reaction was allowed for additional 1 hour, and 10 g of an isopropyl alcohol solution of 3.05 g (15 mol %) of the compound (S-9) was added dropwise over 30 min. Thereafter, the reaction was allowed for additional 1 hour. The mixture was cooled to no higher than 30° C. to obtain copolymerization liquid. The obtained copolymerization liquid was concentrated to 150 g and was transferred to a separatory funnel. 50 g of methanol and 600 g of n-hexane were charged in the separatory funnel, and separation and purification were carried out. After the separation, an under layer liquid was recovered. The under layer liquid was diluted to 100 g with isopropyl alcohol and was transferred to the separatory funnel again. 50 g of methanol and 600 g of n-hexane were charged in the separatory funnel, and separation and purification were carried out. After the separation, an under layer liquid was recovered. The solvent in the recovered under layer liquid was substituted with 4-methyl-2-pentanol, and the total amount was adjusted to 250 g. After the adjustment, 250 g of water was added, and separation and purification were carried out. Following the separation, an upper layer liquid was recovered.

The solvent in the recovered upper layer liquid was substituted with 4-methyl-2-pentanol to form a resin solution. The copolymer contained in the obtained resin solution had the Mw of 9,760 and the Mw/Mn of 1.51, and the yield of the copolymer contained in the obtained resin solution was 65%. In addition, the contents (mol %) of the repeating units derived from the compound (S-8) and the compound (S-9) were 95 and 5, respectively, and content of a fluorine atom was 36.8%. The copolymer is defined as a polymer for an upper layer film (2).

### Preparation of Upper Layer Film-Forming Composition (H)

An upper layer film-forming composition (H) was prepared by mixing 7 parts of the polymer for an upper layer film (1) prepared in Synthesis Example 6, 93 parts of the polymer for an upper layer film (2) prepared in Synthesis Example 7, 10 parts of diethylene glycol monoethyl ether acetate, 10 parts of 4-methyl-2-hexanol (hereinafter, may be also referred to as “MIBC”) and 90 parts of diisoamyl ether (hereinafter, may be also referred to as “DIRE”).

### Example 6

### Preparation of Radiation-Sensitive Resin Composition (T-1)

A composition solution (T-1) of a radiation-sensitive resin composition was prepared by mixing 100 parts of the polymer (B-1) prepared in Synthesis Example 2, 12.1 parts of the sulfonyl compound (A-1) synthesized in Example 1, 1.5 parts of (D-1) an acid diffusion control agent, 1,800 parts of (E-1) a solvent, 770 parts of (E-2) a solvent and 30 parts of (G-1) an additive.

### Examples 7 to 23

### Preparation of Radiation-Sensitive Resin Compositions (T-2) to (T-18)

Composition solutions (T-2) to (T-18) of each radiation-sensitive resin composition were prepared in a manner similar to Example 5 except that the formulation was as shown in Table 2 or Table 3.

TABLE 2

| | | | | | | | (D) Acid | | | | | | |
|---|---|---|---|---|---|---|---|---|---|---|---|---|---|
| | (A) Sulfonium | | | | | | diffusion | | | | (G) Lactone | | |
| | compound | | (B) Polymer | | (C) Polymer | | control agent | | (E) Solvent | | compound | | Type of |
| | | Amount | | Amount | | Amount | | Amount | | Amount | | Amount | sensitive |
| | | blended | | blended | | blended | | blended | | blended | | blended | resin |
| | Type | (parts) | Type | (parts) | Type | (parts) | Type | (parts) | Type | (parts) | Type | (parts) | composition |
| Example 6 | A-1 | 12.1 | B-1 | 100 | — | — | D-1 | 1.5 | E-1 | 1,800 | G-1 | 30 | T-1 |
| | | | | | | | | | E-2 | 770 | | | |
| Example 7 | A-2 | 11.7 | B-1 | 100 | — | — | D-1 | 1.5 | E-1 | 1,800 | G-1 | 30 | T-2 |
| | | | | | | | | | E-2 | 770 | | | |
| Example 8 | A-3 | 11.8 | B-1 | 100 | — | — | D-2 | 7 | E-1 | 1,800 | G-1 | 30 | T-3 |
| | | | | | | | | | E-2 | 770 | | | |
| Example 9 | A-4 | 14.2 | B-1 | 100 | — | — | D-2 | 7 | E-1 | 1,800 | G-1 | 30 | T-4 |
| | | | | | | | | | E-2 | 770 | | | |
| Example 10 | A-1 | 12.1 | B-1 | 100 | C-1 | 5 | D-1 | 1.5 | E-1 | 1,800 | — | — | T-5 |
| | | | | | | | | | E-2 | 770 | | | |
| Example 11 | A-2 | 11.7 | B-1 | 100 | C-1 | 5 | D-1 | 1.5 | E-1 | 1,800 | — | — | T-6 |
| | | | | | | | | | E-2 | 770 | | | |
| Example 12 | A-3 | 11.8 | B-1 | 100 | C-1 | 5 | D-2 | 7 | E-1 | 1,800 | — | — | T-7 |
| | | | | | | | | | E-2 | 770 | | | |
| Example 13 | A-4 | 14.2 | B-1 | 100 | C-1 | 5 | D-2 | 7 | E-1 | 1,800 | — | — | T-8 |
| | | | | | | | | | E-2 | 770 | | | |
| Example 14 | A-1 | 12.1 | B-1 | 100 | C-2 | 5 | D-1 | 1.5 | E-1 | 1,800 | G-1 | 30 | T-9 |
| | | | | | | | | | E-2 | 770 | | | |
| Example 15 | A-2 | 11.7 | B-1 | 100 | C-2 | 5 | D-1 | 1.5 | E-1 | 1,800 | G-1 | 30 | T-10 |
| | | | | | | | | | E-2 | 770 | | | |

TABLE 3

| | | | | | | | (D) Acid | | | | | | |
|---|---|---|---|---|---|---|---|---|---|---|---|---|---|
| | (A) Sulfonium | | | | | | diffusion | | | | (G) Lactone | | Type of |
| | compound | | (B) Polymer | | (C) Polymer | | control agent | | (E) Solvent | | compound | | radiation- |
| | | Amount | | Amount | | Amount | | Amount | | Amount | | Amount | sensitive |
| | | blended | | blended | | blended | | blended | | blended | | blended | resin |
| | Type | (parts) | Type | (parts) | Type | (parts) | Type | (parts) | Type | (parts) | Type | (parts) | composition |
| Example 16 | A-3 | 11.8 | B-1 | 100 | C-2 | 5 | D-2 | 7 | E-1 | 1,800 | G-1 | 30 | T-11 |
| | | | | | | | | | E-2 | 770 | | | |
| Example 17 | A-4 | 14.2 | B-1 | 100 | C-2 | 5 | D-2 | 7 | E-1 | 1,800 | G-1 | 30 | T-12 |
| | | | | | | | | | E-2 | 770 | | | |
| Example 18 | A-1 | 12.1 | B-1 | 100 | C-3 | 5 | D-1 | 1.5 | E-1 | 1,800 | G-1 | 30 | T-13 |
| | | | | | | | | | E-2 | 770 | | | |
| Example 19 | A-2 | 11.7 | B-1 | 100 | C-3 | 5 | D-1 | 1.5 | E-1 | 1,800 | G-1 | 30 | T-14 |
| | | | | | | | | | E-2 | 770 | | | |
| Example 20 | A-3 | 11.8 | B-1 | 100 | C-3 | 5 | D-2 | 7 | E-1 | 1,800 | G-1 | 30 | T-15 |
| | | | | | | | | | E-2 | 770 | | | |
| Example 21 | A-4 | 14.2 | B-1 | 100 | C-3 | 5 | D-2 | 7 | E-1 | 1,800 | G-1 | 30 | T-16 |
| | | | | | | | | | E-2 | 770 | | | |
| Example 22 | A-5 | 15.3 | B-1 | 100 | — | — | D-1 | 1.5 | E-1 | 1,800 | G-1 | 30 | T-17 |
| | | | | | | | | | E-2 | 770 | | | |
| Example 23 | A-5 | 15.3 | B-1 | 100 | C-1 | 5 | D-2 | 7 | E-1 | 1,800 | — | — | T-18 |
| | | | | | | | | | E-2 | 770 | | | |

It is to be noted that each component used in Examples is described below.

### Acid Diffusion Control Agent

(D-1): a compound represented by the following formula (D-1)

(D-1): a compound represented by the following formula (D-2)

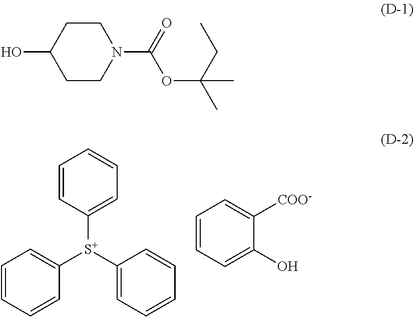

### Solvent (E)

(E-1): propylene glycol monomethyl ether acetate

(E-2): cyclohexanone

### Lactone Compound (G)

(G-1): γ-butyrolactone

Pattern Forming Method (P-1) An under layer antireflective film having a film thickness of 77 nm was formed on the surface of a silicon wafer having a diameter of 8 inches using an under layer antireflective film-forming agent (trade name “ARC29A”, manufactured by Nissan Chemical Industries, Ltd.). A radiation-sensitive resin composition was applied on the surface of the substrate by spin coating. SB (Soft Baking) was carried out on a hot plate at 100° C. for 60 sec to form a photoresist film having a film thickness of 120 nm.

The photoresist film was exposed through a mask pattern using a full field stepper (trade name “NSRS306C”, manufactured by Nikon Corporation). Thereafter, PEB was carried out at 100° C. for 60 sec, then the photoresist film was developed with a 2.38% tetramethylammonium hydroxide aqueous solution (hereinafter, may be also referred to as “TMAH aqueous solution”) at 25° C. for 60 sec, followed by washing with water and drying to form a positive type resist pattern. It is to be noted that the positive type resist pattern was of a 1:1 line-and-space having a line width of 90 nm formed through a mask for forming a 1:1 line-and-space having a target dimension of 90 nm. A scanning electron microscope (trade name “S9380”, manufactured by Hitachi High-Technologies Corporation) was used in line-width measurement of the resist pattern formed by the method. The pattern forming method is designated as (P-1).

### Pattern Forming Method (P-2)

A photoresist film was formed having a film thickness of 75 nm was formed by the radiation-sensitive resin composition on a silicon wafer having a diameter of 12 inches on which an under layer antireflective film pattern had been formed in a similar manner to the pattern forming method (P-1), and soft-baking (SB) was carried out at 120° C. for 60 sec. Next, the composition for upper layer film forming (H) was spin coated on the photoresist film formed, followed by carrying out PB (90° C., 60 sec) to form an upper layer film having a film thickness of 90 nm. Thereafter, the photoresist film was to exposed through a mask pattern using an ArF excimer laser Immersion Scanner (trade name “NSR S610C”, manufactured by Nikon Corporation), under a condition including NA of 1.3, a ratio of 0.800, and Annular. After the exposure, post-exposure baking (PEB) was carried out at 95° C. for 60 sec. Thereafter, the photoresist was developed with a 2.38% TMAH aqueous solution, followed by washing with water and drying to form a positive type resist pattern. It is to be noted that the positive type resist pattern was of a 1:1 line-and-space having a line width of 50 nm formed through a mask for forming a pattern whose target dimension was a line of 50 nm and a pitch of 100 nm. A scanning electron microscope (trade name “CG-4000”, manufactured by Hitachi High-Technologies Corporation) was used for measurement of line-width of the resist pattern formed in the method. The pattern forming method is designated as (P-2).

### Pattern Forming Method (P-3)

A photoresist film having a film thickness of 75 nm was formed by the radiation-sensitive resin composition on a silicon wafer having a diameter of 12 inches on which an under layer antireflective film had been formed in a similar manner to the pattern forming method (P-1), and soft-baking (SB) was carried out at 120° C. for 60 sec. Next, the photoresist film was exposed through a mask pattern using the aforementioned ArF excimer laser Immersion Scanner under a condition including NA of 1.3, a ratio of 0.800, and Annular. After the exposure, post-exposure baking (PEB) was carried out at 95° C. for 60 sec. Thereafter, the photoresist was developed by a 2.38% TMAH aqueous solution, followed by washing with water and drying to form a positive type resist pattern. It is to be noted that the positive type resist pattern was of a 1:1 line-and-space having a line width of 50 nm formed through a mask for forming a pattern whose target dimension was a line of 50 nm and a pitch of 100 nm. A scanning electron microscope (“CG-4000”, manufactured by Hitachi High-Technologies Corporation) was used for measurement of line-width of the resist pattern formed in the method. The pattern forming method is designated as (P-3).

### Example 24

### Formation of Resist Pattern

A resist pattern was formed by the pattern forming method (P-1) using the radiation-sensitive resin composition (T-1) prepared in Example 6. Evaluations of pattern configuration and scum on the resist pattern formed were made according to a method shown below. The results are shown together in Table 4.

### Examples 25 to 46

### Formation of Resist Pattern

A resist pattern was formed in a similar manner to Example 24 except that the radiation-sensitive resin composition and the pattern forming method were as shown in Table 4. It is to be noted that evaluations on development defects when used in liquid immersion lithography was made using a method shown in Development Defect 2 for Examples 28 to 31 and Example 45, or a method shown in Development Defect 1 for Examples 32 to 43 and Example 46. Results of these evaluations are shown in Table 4 together with the evaluation results of the pattern configuration and scum.

Pattern Configuration: Cross sections of the resist pattern formed on the photoresist film were observed by a cross is section-observing SEM (trade name “S4800”, manufactured by Hitachi, Ltd.). Rectangular resist patterns were evaluated as “A (favorable)” and resist patterns having a round upper part were evaluated as “B (poor)”.
Evaluation of Scum: Cross sections of the resist pattern were observed by the aforementioned cross section-observing SEM. In the case where an undissolved matter of the radiation-sensitive resin composition was observed on the substrate, the evaluation was made as “B (poor)”, whereas in the case where an undissolved matter of radiation-sensitive resin composition was not observed on the substrate, the evaluation was made as “A (favorable)”.
Development Defect 1: First, a coating having a film thickness of 110 nm was formed by the radiation-sensitive resin composition on a silicon wafer having a diameter of 12 inches on which the aforementioned under layer antireflective film had been formed, and soft-baking (SB) was carried out at 110° C. for 60 sec. Next, the coating was exposed through a mask pattern using the aforementioned ArF excimer laser Immersion Scanner, under a condition including NA of 1.3, a ratio of 0.800, and Dipole. After the exposure, post-baking (PEB) was carried out at 95° C. for 60 sec. Thereafter, the photoresist film was developed with a 2.38% tetramethylammonium hydroxide aqueous solution, followed by washing with water and drying to form a positive type resist pattern. In this process, an exposure dose by which a line-and-space pattern having a width of 45 nm was formed is defined as an optimal exposure dose. A line-and-space pattern having a line width of 45 nm was formed on the entire surface of the wafer with the optimal exposure dose, and the wafer was employed as a wafer for inspection of defects. It is to be noted that a scanning electron microscope (trade name “CC-4000”, manufactured by Hitachi High-Technologies Corporation) was used for the measurement of line-width.

Thereafter, the number of defects on the wafer for inspection of defects was counted using a high resolution wafer defect measurement apparatus (trade name “KLA2810”, manufactured by KLA-Tencor). Furthermore, the counted defects were classified into the defects judged to be derived from the resist, and those resulting from foreign substances derived from the outside. After the classification, in the case where a total number of defects judged to be derived from the resist (number of defects) was no less than 100/wafer, the evaluation was made as “C (somewhat favorable)”, in the case where a total number of defects judged to be derived from the resist (number of defects) was no less than 50/wafer, and in the case where a total number of defects judged to be derived from the resist (number of defects) was no greater than 100/wafer, the evaluation was made as “B (favorable)”, and in the case where a total number of defects judged to be derived from the resist (number of defects) was no greater than 50/wafer, the evaluation was made as “A (very favorable)”.

Development Defect 2: A wafer for inspecting defects was formed in a similar manner to Development Defect 1 except that the composition for forming an upper layer film (H) was spin coated on a coating which had been formed by the radiation-sensitive resin composition followed by carrying out PB at 90° C. for 60 sec to form an upper layer film having a film thickness of 90 nm.

Thereafter, the number of defects on the wafer for inspection of defects was counted using the high resolution wafer defect measurement apparatus described above. Furthermore, the counted defects were classified into the defects judged to be derived from the resist, and those resulting from foreign substances derived from the outside. After the classification, in the case where a total number of defects judged to be derived from the resist (number of defects) was no less than 100/wafer, the evaluation was made as “C (somewhat favorable)”, in the case where a total number of defects judged to be derived from the resist (number of defects) was no less than 50/wafer, and in the case where a total number of defects judged to be derived from the resist (number of defects) was no greater than 100/wafer, the evaluation was made as “B (favorable)”, and in the case where a total number of defects judged to be derived from the resist (number of defects) was no greater than 50/wafer, the evaluation was made as “A (very favorable)”.

TABLE 4

| | Type of | | | | | |
|---|---|---|---|---|---|---|
| | radiation- | | Evaluation | | | |
| | sensitive | Pattern | of cross- | | | |
| | resin | forming | sectional | Evaluation | Development | Development |
| | composition | method | shape | of scum | defect 1 | defect 2 |
| Example 24 | T-1 | P-1 | A | A | — | — |
| Example 25 | T-2 | P-1 | A | A | — | — |
| Example 26 | T-3 | P-1 | A | A | — | — |
| Example 27 | T-4 | P-1 | A | A | — | — |
| Example 28 | T-1 | P-2 | A | A | — | A |
| Example 29 | T-2 | P-2 | A | A | — | A |
| Example 30 | T-3 | P-2 | A | A | — | A |
| Example 31 | T-4 | P-2 | A | A | — | A |
| Example 32 | T-5 | P-3 | A | A | A | — |
| Example 33 | T-6 | P-3 | A | A | A | — |
| Example 34 | T-7 | P-3 | A | A | A | — |
| Example 35 | T-8 | P-3 | A | A | A | — |
| Example 36 | T-9 | P-3 | A | A | A | — |
| Example 37 | T-10 | P-3 | A | A | A | — |
| Example 38 | T-11 | P-3 | A | A | A | — |
| Example 39 | T-12 | P-3 | A | A | A | — |
| Example 40 | T-13 | P-3 | A | A | A | — |
| Example 41 | T-14 | P-3 | A | A | A | — |
| Example 42 | T-15 | P-3 | A | A | A | — |
| Example 43 | T-16 | P-3 | A | A | A | — |
| Example 44 | T-17 | P-1 | B | B | — | — |
| Example 45 | T-17 | P-2 | B | B | — | C |
| Example 46 | T-18 | P-3 | B | B | C | — |

The radiation-sensitive resin compositions prepared in Examples 6 to 9 were excellent in rectangularity in the cross-sectional shape of the resist pattern after development and also caused no scum (Examples 24 to 27). In addition, the radiation-sensitive resin compositions prepared in Examples 6 to 9 were less likely to cause development defects also when used in liquid immersion lithography after forming the upper layer film (Examples 28 to 31). Furthermore, the radiation-sensitive resin compositions prepared in Examples 10 to 21 were excellent in rectangularity in cross-sectional shape of the resist pattern obtained after development and also caused no scum, and were less likely to cause development defects even if used in liquid immersion lithography without forming the upper layer film (Examples 32 to 43). On the other hand, the radiation-sensitive resin composition prepared in Example 22 gave less rectangular cross-sectional shape of the resist pattern after development than the radiation-sensitive resin compositions prepared in Examples 10 to 21 and caused scum, but was satisfactory for use as a radiation-sensitive resin composition (Example 44). In addition, in the case where the radiation-sensitive resin composition prepared in Example 22 was used in liquid immersion lithography after forming the upper layer film, more development defects were caused than the case of the radiation-sensitive resin compositions prepared in Examples 10 to 21, but the radiation-sensitive resin composition prepared in Example 22 was able to be used as a radiation-sensitive resin composition (Example 45). Furthermore, a radiation-sensitive resin composition prepared in Example 23 had no rectangular cross-sectional shape of the resist pattern after development, caused scum and was likely to cause more development defects in use in liquid immersion lithography without forming upper layer films than the radiation-sensitive resin composition prepared in Examples 10 to 21, but was satisfactory for use as a radiation-sensitive resin composition (Example 46).

The radiation-sensitive resin compositions of the embodiment of the present invention can be suitably utilized in semiconductor producing processes which require further miniaturization of patterns in the future.

Obviously, numerous modifications and variations of the present invention are possible in light of the above teachings. It is therefore to be understood that within the scope of the appended claims, the invention may be practiced otherwise than as specifically described herein.

1. A radiation-sensitive resin composition comprising:
a sulfonium compound represented by a following general formula (1); and
a first polymer that serves as a base resin, 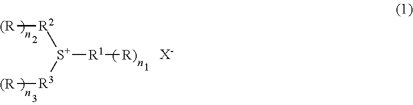
wherein, in the above general formula (1),
R¹ represents an aromatic hydrocarbon group having a valency of (n₁+1) and 6 to 30 carbon atoms, an aliphatic chain hydrocarbon group having a valency of (n₁+1) and 1 to 10 carbon atoms or an alicyclic hydrocarbon group having a valency of (n₁+1) and 3 to 10 carbon atoms;
R² represents an aromatic hydrocarbon group having a valency of (n₂+1) and 6 to 30 carbon atoms, an aliphatic chain hydrocarbon group having a valency of (n₂+1) and 1 to 20 carbon atoms or an alicyclic hydrocarbon group having a valency of (n₂+1) and 3 to 20 carbon atoms;
R³ represents an aromatic hydrocarbon group having a valency of (n₃+1) and 6 to 30 carbon atoms, an aliphatic chain hydrocarbon group having a valency of (n₃+1) and 1 to 30 carbon atoms or an alicyclic hydrocarbon group having a valency of (n₃+1) and 3 to 30 carbon atoms,
wherein two among R¹ to R³ are optionally bonded with one another to form a cyclic structure including a sulfur cation,
wherein a part or all of hydrogen atoms R¹ to R³ have are unsubstituted or substituted;
R represents a group represented by a following general formula (2), wherein in a case where R is present in a plurality of number, Rs present in a plurality of number are each independent;
n₁ to n₃ each independently represent an integer of 0 to 5, wherein n₁+n₂+n₃≧1; and
X⁻ represents an anion;
-A-R⁴ (2)
wherein, in the above general formula (2),
R⁴ represents an alkali-dissociable group; and
A represents an oxygen atom, a —NR⁵— group, a —CO—O—* group or a —SO₂—O—* group, wherein
R⁵ represents a hydrogen atom or an alkali-dissociable group; and
is “*” denotes a binding site to R⁴.

2. The radiation-sensitive resin composition according to claim 1, wherein the sulfonium compound is a compound represented by a following general formula (1-1): 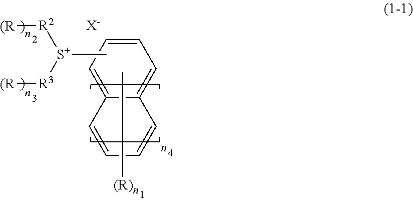
wherein, in the above general formula (1-1),
R², R³, R, n₁ to n₃ and X⁻ are as defined in the above general formula (1), wherein R² and R³ are optionally bonded with one another to form a cyclic structure including a sulfur cation; and
n₄ represents 0 or 1.

3. The radiation-sensitive resin composition according to claim 1, wherein the sulfonium compound is a compound represented by a following general formula (1-1a): 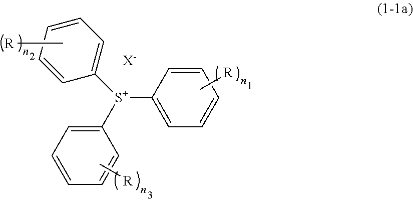
wherein, in the above general formula (1-1a), R, n₁ to n₃ and X⁻ are as defined in the above general formula (1).

4. The radiation-sensitive resin composition according to claim 1, wherein at least one R in the sulfonium compound is a group represented by a following general formula (2a): 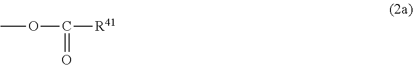
wherein, in the above general formula (2a), R⁴¹ represents a hydrocarbon group having 1 to 7 carbon atoms, wherein a part or all of hydrogen atoms are substituted with a fluorine atom, wherein in a case where R represented by the above general formula (2a) is present in a plurality of number, R⁴¹s present in a plurality of number are each independent.

5. The radiation-sensitive resin composition according to claim 1, further comprising a second polymer having a fluorine atom.

6. The radiation-sensitive resin composition according to claim 5, wherein an amount of the second polymer blended is 0.1 to 20 parts by mass with respect to 100 parts by mass of the first polymer.

7. A method for forming a resist pattern, comprising:
forming a photoresist film on a substrate using the radiation-sensitive resin composition according to claim 1;
is exposing the photoresist film; and
developing the exposed photoresist film to form a resist pattern.

8. The method for forming a resist pattern according to claim 7, wherein liquid immersion lithography of the photoresist film is carried out when the photoresist film is exposed.

9. A sulfonium compound represented by a following general formula (1): 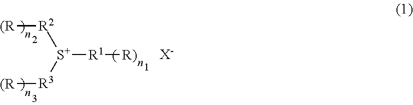
wherein, in the general formula (1),
R¹ represents an aromatic hydrocarbon group having a valency of (n₁+1) and 6 to 30 carbon atoms, an aliphatic chain hydrocarbon group having a valency of (n₁+1) and 1 to 10 carbon atoms or an alicyclic hydrocarbon group having a valency of (n₁+1) and 3 to 10 carbon atoms;
R² represents an aromatic hydrocarbon group having a valency of (n₂+1) and 6 to 30 carbon atoms, an aliphatic chain hydrocarbon group having a valency of (n₂+1) and 1 to 20 carbon atoms or an alicyclic hydrocarbon group having a valency of (n₂+1) and 3 to 20 carbon atoms;
R³ represents an aromatic hydrocarbon group having a valency of (n₃+1) and 6 to 30 carbon atoms, an aliphatic chain hydrocarbon group having a valency of (n₃+1) and 1 to 30 carbon atoms, or an alicyclic hydrocarbon group having a valency of (n₃+1) and 3 to 30 carbon atoms,
wherein two among R¹ to R³ are optionally bonded with one another to form a cyclic structure including a sulfur cation,
wherein a part or all of hydrogen atoms R¹ to R³ have are unsubstituted or substituted;
R represents a group represented by a following general formula (2), wherein in a case where R is present in a plurality of number, Rs present in a plurality of number are each independent;
n₁ to n₃ each independently represent an integer of 0 to 5, wherein n₁+n₂+n₃ 1; and
X⁻ represents an anion;
-A-R⁴ (2)
wherein, in the general formula (2),
R⁴ represents an alkali-dissociable group; and
A represents an oxygen atom, a —NR⁵— group, a —CO—O—* group or a —SO₂—O—* group, wherein
R⁵ represents a hydrogen atom or an alkali-dissociable group; and
“*” denotes a binding site to R⁴.

10. The sulfonium compound according to claim 9, the sulfonium compound being represented by a following general formula (1-1): 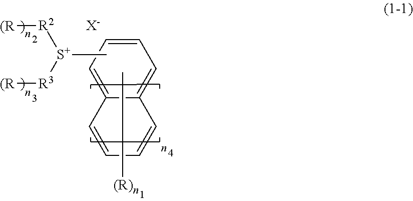
wherein, in the general formula (1-1),
R², R³, R, n₁ to n₃ and X⁻ are as defined in the above general formula (1), wherein R² and R³ are optionally bonded with one another to form a cyclic structure including a sulfur cation;
and n₄ represents 0 or 1.

11. The sulfonium compound according to claim 9, the sulfonium compound being represented by a following general formula (1-1a): 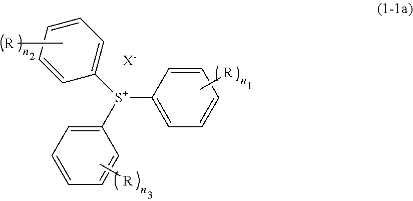
wherein, in the general formula (1-1a), R, n₁ to n₃ and X⁻ are as defined in the above general formula (1).

12. The sulfonium compound according to claim 9, wherein at least one R in the sulfonium compound is a group represented by a following general formula (2a): 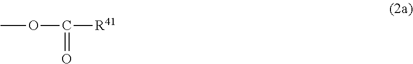
wherein, in the general formula (2a), R⁴¹ represents a hydrocarbon group having 1 to 7 carbon atoms, wherein a part or all of hydrogen atoms are substituted with a fluorine atom, wherein in a case where R represented by the above general formula (2a) is present in a plurality of number, R⁴¹s present in a plurality of number are each independent.
